(19) United States
(12) Patent Application Publication
(10) Pub. No.: US 2009/0170009 A1
(43) Pub. Date: Jul. 2, 2009

(54) HOLOGRAPHIC RECORDING COMPOSITION AND HOLOGRAPHIC RECORDING MEDIUM

(75) Inventors: Satoru YAMADA, Kanagawa (JP); Hiroyuki SUZUKI, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/338,382

(22) Filed: Dec. 18, 2008

(30) Foreign Application Priority Data
Dec. 27, 2007 (JP) 2007-336110

(51) Int. Cl. G03F 7/028; C07C 255/01; C07C 69/76

(52) U.S. Cl. 430/2; 558/401; 560/76

(57) ABSTRACT

The present invention provides a holographic recording composition comprising a compound denoted by general formula (I) and a holographic recording medium comprising a recording layer, wherein the recording layer comprises a compound denoted by general formula (I).

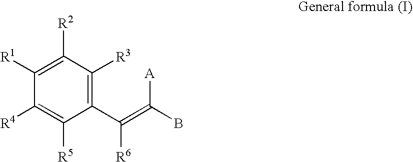

In general formula (I), R¹ denotes a hydrogen atom or a substituent having a Hammett value, σp, of equal to or greater than −0.30, each of R², R³, R⁴, R⁵, and R⁶ independently denotes a hydrogen atom or a substituent, each of A and B independently denotes a substituent and a combination of A and B is (cyano group, cyano group), (oxycarbonyl group, oxycarbonyl group), (acyl group, acyl group), (sulfonyl group, sulfonyl group), (cyano group, acyl group), (cyano group, sulfonyl group), (oxycarbonyl group, acyl group), (oxycarbonyl group, sulfonyl group), or (sulfonyl group, acyl group), and at least one of R¹, R², R³, R⁴, R⁵, R⁶, A, and B comprises a polymerizable group.

### CROSS-REFERENCE TO RELATED APPLICATIONS

This application claims the benefit of priority under 35 USC 119 to Japanese Patent Application No. 2007-336110 filed on Dec. 27, 2007, which is expressly incorporated herein by reference in its entirety.

### BACKGROUND OF THE INVENTION

1. Field of the Invention

The present invention relates to a holographic recording composition comprising an arylidene compound, and more specifically, to a holographic recording composition that is suited to the manufacturing of a holographic recording medium permitting the writing of information with a 405 nm laser, for example, and that is particularly suited to the manufacturing of a volume holographic recording medium having a relatively thick recording layer. The present invention further relates to a holographic recording medium comprising a recording layer comprising the above arylidene compound.

2. Discussion of the Background

Holographic optical recording media based on the principle of the holograph have been developed. Recording of information on holographic optical recording media is carried out by superposing an informing light containing image information and a reference light in a recording layer comprised of a photosensitive composition to write an interference fringe thus formed in the recording layer. During the reproduction of information, a reference light is directed at a prescribed angle into the recording layer in which the information has been recorded, causing optical diffraction of the reference light by the interference fringe which has been formed, reproducing the informing light. For example, Published Japanese Translation of a PCT International Application (TOKUHYO) No. 2005-502918 or English language family member WO 03/023519, US2003/0087104 A1 and U.S. Pat. No. 6,765,061, which are expressly incorporated herein by reference in their entirety, disclose the use of a urethane matrix and a phenyl acrylate derivative in a holographic recording medium of the photopolymer type.

In recent years, volume holography, and, more particularly, digital volume holography, have been developed to practical levels for ultrahigh-density optical recording and have been garnering attention. Volume holography is a method of writing interference fringes three-dimensionally by also actively utilizing the direction of thickness of an optical recording medium. It is advantageous in that increasing the thickness permits greater diffraction efficiency and multiplexed recording increases the recording capacity. Digital volume holography is a computer-oriented holographic recording method in which the image data being recorded are limited to a binary digital pattern while employing a recording medium and recording system similar to those of volume holography. In digital volume holography, for example, image information such as an analog drawing is first digitized and then expanded into two-dimensional digital pattern information, which is recorded as image information. During reproduction, the digital pattern information is read and decoded to restore the original image information, which is displayed. Thus, even when the signal-to-noise (S/N) ratio deteriorates somewhat during reproduction, by conducting differential detection or conducting error correction by encoding the two-dimensional data, it is possible to reproduce the original data in an extremely faithful manner (see Japanese Unexamined Patent Publication (KOKAI) Heisei No. 11-311936 or English language family member US 2002/0114027 A1, which are expressly incorporated herein by reference in their entirety).

Further increases in the recording capacity of the above volume holographic optical recording medium are required. For example, Japanese Unexamined Patent Publication (KOKAI) No. 2005-275158, US 2005/233246A1, and Japanese Unexamined Patent Publication (KOKAI) No. 2007-272044, which are expressly incorporated herein by reference in their entirety, disclose recording media incorporating recording monomers in the form of dye compounds to increase recording capacity.

In recent years, the wavelength of recording lights has tended to become shorter to increase recording capacity. The use of recording lights with wavelengths of about 400 nm, specifically 405 nm, has begun. However, dye compounds with high absorption in the visible light range are employed in the recording media described in Japanese Unexamined Patent Publication (KOKAI) No. 2005-275158, US 2005/233246A1, and Japanese Unexamined Patent Publication (KOKAI) No. 2007-272044, resulting in a decrease in transmittance of the medium at wavelengths of about 400 nm. Thus, it is difficult to conduct high-sensitivity recording with a recording light with a wavelength of about 400 nm.

### SUMMARY OF THE INVENTION

An aspect of the present invention provides for a holographic recording composition that is suited to digital volume holography, and affords high sensitivity and a large recording capacity in recording with light of short wavelengths, and a holographic recording medium permitting ultrahigh-density optical recording.

As a result of extensive research, the present inventors discovered that a holographic recording medium permitting high-density and high-sensitivity in recording with light of short wavelengths was obtained by means of an arylidene compound denoted by general formula (I); the present invention was devised on that basis.

An aspect of the present invention relates to a holographic recording composition comprising a compound denoted by general formula (I).

A further aspect of the present invention relates to a holographic recording medium comprising a recording layer, wherein the recording layer comprises a compound denoted by general formula (I).

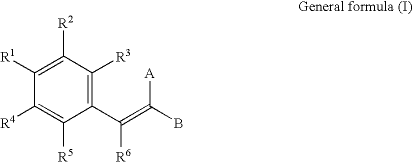

In general formula (I), R¹ denotes a hydrogen atom or a substituent having a Hammett value, σp, of equal to or greater than −0.30, each of R², R³, R⁴, R⁵, and R⁶ independently denotes a hydrogen atom or a substituent, each of A and B independently denotes a substituent and a combination of A and B is one of Combinations 1 to 9, and at least one of R¹, R², R³, R⁴, R⁵, R⁶, A, and B comprises a polymerizable group.

| | | |
|---|---|---|
| (Combination 1) | A: cyano group, | B: cyano group; |
| (Combination 2) | A: oxycarbonyl group, | B: oxycarbonyl group; |
| (Combination 3) | A: acyl group, | B: acyl group; |
| (Combination 4) | A: sulfonyl group, | B: sulfonyl group; |
| (Combination 5) | A: cyano group, | B: acyl group; |
| (Combination 6) | A: cyano group, | B: sulfonyl group; |
| (Combination 7) | A: oxycarbonyl group, | B: acyl group; |
| (Combination 8) | A: oxycarbonyl group, | B: sulfonyl group; |
| (Combination 9) | A: sulfonyl group, | B: acyl group. |

In general formula (I), R¹ may denote a hydrogen atom, alkyl group, aryl group, heterocyclic group, acyl group, sulfonyl group, acyloxy group, alkoxycarbonyl group, aryloxycarbonyl group, alkoxy group, aryloxy group, acylamino group, sulfonylamino group, cyano group, or halogen group, each of R², R³, R⁴, and R⁵ may independently denote a hydrogen atom, alkyl group, aryl group, heterocyclic group, acyl group, sulfonyl group, acyloxy group, alkoxycarbonyl group, aryloxycarbonyl group, alkoxy group, aryloxy group, amino group, acylamino group, sulfonylamino group, or halogen group, and R⁶ may denote a hydrogen atom or an alkyl group.

In general formula (I), R¹ may denote an alkyl group, aryl group, alkoxycarbonyl group, acyloxy group, or halogen group.

In general formula (I), each of R², R³, R⁴, and R⁵ may independently denote a hydrogen atom, alkyl group, aryl group, alkoxy group, aryloxy group, amino group, acyl group, acyloxy group, or halogen group.

In general formula (I), the combination of A and B may be Combination 1, 2, 3, 4, 5, 7, or 8.

The polymerizable group may be a radical polymerizable group.

The compound denoted by general formula (I) may have a molar absorbance coefficient of equal to or smaller than 200 mol·l·cm⁻¹ at a wavelength of 405 nm.

The compound denoted by general formula (I) may have a maximum absorption wavelength of shorter than 405 nm.

The holographic recording composition and the recording layer in the holographic recording medium may further comprise a photopolymerization initiator.

The photopolymerization initiator may be a photo-induced radical polymerization initiator, and the photo-induced radical polymerization initiator may be a compound denoted by general formula (II).

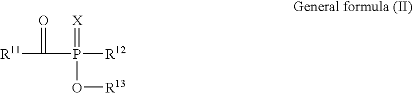

In general formula (II), each of R¹¹, R¹², and R¹³ independently denotes an alkyl group, aryl group, or heterocyclic group, and X denotes an oxygen atom or sulfur atom.

The holographic recording composition and the recording layer in the holographic recording medium may further comprise a polyfunctional isocyanate and a polyfunctional alcohol.

The compound denoted by general formula (I) can permit high-sensitivity recording when employing a recording light source in the form of a laser having a wavelength in the area of 405 nm, specifically a center wavelength of 405±20 nm. It is also suited to digital volume holography, permitting the use of inexpensive lasers and shorter writing times.

The holographic recording medium of the present invention can permit ultrahigh-density optical recording because it comprises a holographic recording layer containing the above compound, and is optimal for volume holography, particularly digital volume holography recording media.

Other exemplary embodiments and advantages of the present invention may be ascertained by reviewing the present disclosure and the accompanying drawings.

### BRIEF DESCRIPTION OF THE DRAWINGS

The present invention will be described in the following text by the exemplary, non-limiting embodiments shown in the figures, wherein:

FIG. 1 is a schematic cross-sectional view of an example of a holographic recording medium according to a first implementation embodiment.

FIG. 2 is a schematic cross-sectional view of an example of a holographic recording medium according to a second implementation embodiment.

FIG. 3 is a drawing descriptive of an example of an optical system permitting recording and reproducing of information on a holographic recording medium.

FIG. 4 is a block diagram showing an example of the overall configuration of a recording and reproducing device suited to use in recording and reproducing information on the holographic recording medium of the present invention.

FIG. 5 is a schematic of the optical system of a planar wave tester.

Explanations of symbols in the drawings are as follows:
1 Lower substrate
2 Reflective film
3 Servo pit pattern
4 Recording layer
5 Upper substrate
6 Filter layer
7 Second gap layer
8 First gap layer
12 Objective lens
13 Dichroic mirror
14 Detector
15 1/4 wavelength plate
16 Polarizing plate
17 Half mirror
20 Holographic recording medium
21 Holographic recording medium
22 Holographic recording medium
31 Pickup
81 Spindle
82 Spindle motor
83 Spindle servo circuit
84 Driving device
85 Detection circuit
86 Focus servo circuit
87 Tracking servo circuit
88 Slide servo circuit
89 Signal processing circuit
90 Controller
91 Operation element
100 Optical recording and reproducing device
A Entry and exit surface
FE Focus error signal
TE Tracking error signal
RF Reproduction signal

### DETAILED DESCRIPTIONS OF THE EMBODIMENTS

The following preferred specific embodiments are, therefore, to be construed as merely illustrative, and non-limiting to the remainder of the disclosure in any way whatsoever. In this regard, no attempt is made to show structural details of the present invention in more detail than is necessary for fundamental understanding of the present invention; the description taken with the drawings making apparent to those skilled in the art how several forms of the present invention may be embodied in practice.

### Holographic Recording Composition

The holographic recording composition comprises an arylidene compound denoted by general formula (I). As set forth above, holographic recording is a method of recording information by superposing an informing light containing information and a reference light in a recording layer to write an interference fringe thus formed in the recording layer. Volume holographic recording is a method of recording information in holographic recording in which a three-dimensional interference image is written in the recording layer. In the present invention, the phrase “holographic recording compound” refers to a compound that permits the recording of an interference fringe as refractive index modulation, either directly or indirectly, by irradiating light to record information. The compound denoted by general formula (I) can undergo a polymerization reaction, either directly or through the action of a photopolymerization initiator, when irradiated with light, thereby permitting the recording of interference fringes as refractive index modulation.

The compound denoted by general formula (I) will be described in greater detail below.

### Compound Denoted by General Formula (I)

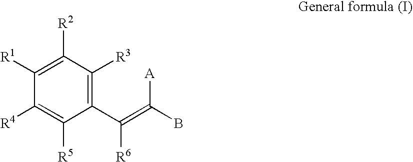

In general formula (I), R¹ denotes a hydrogen atom or a substituent having a Hammett value, σp, of equal to or greater than −0.30. The Hammett σp value is described in detail in Hansch, C., Leo, A., Taft, R. W. Chem. Rev. 1991, 91, 165-195, which is expressly incorporated herein by reference in its entirety. The σp value of a hydrogen atom is 0.00. When the Hammett σp value of R¹ is equal to or greater than −0.30, absorption by the compound at a wavelength of about 400 nm is low, permitting the obtaining of a medium of high transmittance in the above wavelength range. From the perspective of transmittance, the Hammett σp value of the substituent denoted by R¹ is equal to or greater than −0.30, and from the perspective of refractive index, desirably equal to or less than 0.30. The compound denoted by general formula (I) can comprise a polymerizable group in the R¹ moiety, as will be set forth further below. In that case, the Hammett σp value of the substituent is referred to as the Hammett σp value of the R¹ moiety comprising the polymerizable group.

By contrast, a compound with a Hammett σp value for the R¹ moiety of less than −0.30 have considerable absorption at a wavelength of about 405 nm. It is difficult to conduct high-sensitivity recording in the above wavelength region with a medium having a recording layer containing such a compound. For example, the Hammett σp value of amino groups is about −0.34 to −0.9. When the R¹ moiety is an amino group (amino group: σp value=−0.66, methylamino group: σp value=−0.70, ethylamino group: σp value=−0.61, dimethylamino group: σp value=−0.83, diethylamino group: σp value=−0.72, hydroxyamino group: σp value=−0.34), the compound absorbs markedly longer wavelengths, and absorption at wavelengths of about 400 nm increases. As will be described further below in the form of a comparative example, when a dialkylamino group is substituted with the polymerizable group, for example, the molar absorbance coefficient of the compound at a wavelength of 405 nm exceeds 10,000 mol·l·cm⁻¹. Thus, the transmittance of the medium at the above wavelength drops sharply, greatly compromising recording sensitivity.

R¹ is desirably a hydrogen atom (σp value=0.00), alkyl group (methyl group: σp value=−0.17, ethyl group: σp value=0.00), aryl group (phenyl group: σp value=−0.01, tolyl group: σp value=−0.08), heterocyclic group (4-pyridyl group: σp value=0.44), acyl group (formyl group: σp value=0.42, acetyl group: σp value=0.06), sulfonyl group (methylsulfonyl group: σp value=0.36), acyloxy group, alkoxycarbonyl group (methoxycarbonyl group: σp value=0.31), aryloxycarbonyl group (phenoxycarbonyl group: σp value=0.44), alkoxy group (methoxy group: σp value=−0.27, ethoxy group: σp value=−0.24), aryloxy group, acylamino group (methylacylamino group: σp value=0.00), sulfonylamino group (methylsulfonylamino group: σp value=0.03), cyano group (σp value=0.66), or halogen group (chloro group: σp value=0.23, bromo group: σp value=0.23, iodo group: σp value=0.18); and is preferably a hydrogen atom, alkyl group, aryl group, alkoxycarbonyl group, acyloxy group, or halogen group.

In general formula (I), each of R², R³, R⁴, R⁵, and R⁶ independently denotes a hydrogen atom or a substituent. The substituent is not specifically limited. Alkyl groups, aryl groups, heterocyclic groups, acyl groups, sulfonyl groups, acyloxy groups, alkoxycarbonyl groups, aryloxycarbonyl groups, alkoxy groups, aryloxy groups, amino groups, acylamino groups, sulfonylamino groups, and halogen groups are suitable. Each of R², R³, R⁴, and R⁵ desirably independently denotes a hydrogen atom or one of the above suitable substituents. R⁶ desirably denotes a hydrogen atom or alkyl group. Each of R², R³, R⁴, and R⁵ preferably independently denotes a hydrogen atom, alkyl group, aryl group, alkoxy groups, aryloxy group, amino group, acyl group, acyloxy groups, or halogen group.

The above substituents will be described in greater detail below.

The alkyl groups denoted by R¹, R², R³, R⁴, R⁵, and R⁶ above can be linear or branched, substituted or unsubstituted. They desirably comprise 1 to 10 carbon atoms, preferably 1 to 6 carbon atoms, and more preferably, 1 to 4 carbon atoms. In the present invention, the “number of carbon atoms” of a given group means the number of carbon atoms of the portion excluding the substituent for a group having a substituent.

Specific examples of the alkyl groups are methyl, ethyl, normal propyl, isopropyl, normal butyl, isobutyl, tertiary butyl, pentyl, cyclopentyl, hexyl cyclohexyl, heptyl, octyl, tertiary octyl, 2-ethylhexyl, decyl, dodecyl, and octadecyl groups.

The aryl groups denoted by R¹, R², R³, R⁴, R⁵, and R⁶ above are not specifically limited, and can be suitably selected based on the objective. Aryl groups having 6 to 20 carbon atoms are desirable, those having 6 to 10 carbon atoms are preferred, and those having 6 carbon atoms are particularly preferred. Specific examples are phenyl, tolyl, naphthyl, and anthranyl groups.

The heterocyclic groups denoted by R¹, R², R³, R⁴, R⁵, and R⁶ above are not specifically limited, and can be suitably selected based on the objective. Heterocyclic groups having 4 to 20 carbon atoms are desirable, those having 4 to 10 carbon atoms are preferred, and those having 4 or 5 carbon atoms are of even greater preference. Specific examples are pyridyl, piperidyl, piperazyl, pyrrole, and morpholino groups.

The acyl groups denoted by R¹, R², R³, R⁴, R⁵, and R⁶ above desirably have 1 to 10 carbon atoms, preferably 1 to 6 carbon atoms, and more preferably, 1 to 4 carbon atoms. Specific examples are methylcarbonyl groups, ethylcarbonyl groups, normal propylcarbonyl groups, isopropylcarbonyl groups, normal butylcarbonyl groups, isobutyl carbonyl groups, tertiary butylcarbonyl groups, pentylcarbonyl groups, cyclopentylcarbonyl groups, hexylcarbonyl groups, cyclohexylcarbonyl groups, heptylcarbonyl groups, octylcarbonyl groups, tertiary octylcarbonyl groups, 2-ethylhexylcarbonyl groups, decylcarbonyl groups, dodecylcarbonyl groups, and benzoyl groups.

The sulfonyl groups denoted by R¹, R², R³, R⁴, R⁵, and R⁶ above desirably have 1 to 10 carbon atoms, preferably 1 to 6 carbon atoms, and more preferably 1 to 4 carbon atoms. Specific examples are methylsulfonyl groups, ethylsulfonyl groups, normal propylsulfonyl groups, isopropylsulfonyl groups, normal butylsulfonyl groups, isobutyl sulfonyl groups, tertiary butylsulfonyl groups, pentylsulfonyl groups, cyclopentylsulfonyl groups, hexylsulfonyl groups, cyclohexylsulfonyl groups, heptylsulfonyl groups, octylsulfonyl groups, tertiary octylsulfonyl groups, 2-ethylhexylsulfonyl groups, decylsulfonyl groups, and dodecylsulfonyl groups.

The acyloxy groups denoted by R¹, R², R³, R⁴, R⁵, and R⁶ above desirably have 1 to 10 carbon atoms, preferably 1 to 6 carbon atoms, and more preferably 1 to 4 carbon atoms. Specific examples are methylcarbonyloxy groups, ethylcarbonyloxy groups, normal propylcarbonyloxy groups, isopropylcarbonyloxy groups, normal butylcarbonyloxy groups, isobutylcarbonyloxy groups, tertiary butylcarbonyloxy groups, pentylcarbonyloxy groups, cyclopentylcarbonyloxy groups, hexylcarbonyloxy groups, cyclohexylcarbonyloxy groups, heptylcarbonyloxy groups, octylcarbonyloxy groups, tertiary octylcarbonyloxy groups, 2-ethylhexylcarbonyloxy groups, decylcarbonyloxy groups, dodecylcarbonyloxy groups, and benzoyloxy groups.

The alkoxycarbonyl groups denoted by R¹, R², R³, R⁴, R⁵, and R⁶ above desirably have 1 to 10 carbon atoms, preferably 1 to 6 carbon atoms, and more preferably 1 to 4 carbon atoms. Specific examples are methyloxycarbonyl groups, ethyloxycarbonyl groups, normal propyloxycarbonyl groups, isopropyloxycarbonyl groups, normal butyloxycarbonyl groups, isobutyloxycarbonyl groups, tertiary butyloxycarbonyl groups pentyloxycarbonyl groups, cyclopentyloxycarbonyl groups, hexyloxycarbonyl groups, cyclohexyloxycarbonyl groups, heptyloxycarbonyl groups, octyloxycarbonyl groups, tertiary octyloxycarbonyl groups, 2-ethylhexyloxycarbonyl groups, decyloxycarbonyl groups, and dodecyloxycarbonyl groups.

The aryloxycarbonyl groups denoted by R¹, R², R³, R⁴, R⁵, and R⁶ above desirably have 7 to 30 carbon atoms, preferably 7 to 20 carbon atoms. Examples are phenyloxycarbonyl groups, naphthyloxycarbonyl groups, and anthranyloxycarbonyl groups.

The acylamino groups denoted by R¹, R², R³, R⁴, R⁵, and R⁶ above desirably have 1 to 10 carbon atoms, preferably 1 to 6 carbon atoms, and more preferably 1 to 4 carbon atoms. Specific examples are methylcarbonylamino groups, ethylcarbonylamino groups, and phenylcarbonylamino groups.

The sulfonylamino groups denoted by R¹, R², R³, R⁴, R⁵, and R⁶ above desirably have 1 to 10 carbon atoms, preferably 1 to 6 carbon atoms, and more preferably 1 to 4 carbon atoms. Specific examples are methylsulfonylamino groups, ethylsulfonylamino groups, and phenylsulfonylamino groups.

Examples of the halogen groups denoted by R¹, R², R³, R⁴, R⁵, and R⁶ above are chloro, bromo, and iodo groups. Bromo groups are desirable.

The alkoxy groups denoted by R¹, R², R³, R⁴, R⁵, and R⁶ above desirably have 1 to 30 carbon atoms, preferably 1 to 20 carbon atoms. Examples of such alkoxy groups are methoxy groups, ethoxy groups, normal propyloxy groups, isopropyl oxy groups, normal butyloxy groups, isobutyloxy groups, tertiary butyloxy groups, pentyloxy groups, cyclopentyloxy groups, hexyloxy groups, cyclohexyloxy groups, heptyloxy groups, octyloxy groups, tertiary octyloxy groups, 2-ethylhexyloxy groups, decyloxy groups, dodecyloxy groups, octadecyloxy groups, 2,3-dibromopropyloxy groups, adamantyloxy groups, benzyloxy groups, and 4-bromobenzyloxy groups.

The aryloxy groups denoted by R¹, R², R³, R⁴, R⁵, and R⁶ above desirably have 6 to 30 carbon atoms, preferably 6 to 20 carbon atoms. Examples are phenyloxy groups, naphthyloxy groups, and anthranyloxy groups.

The amino groups denoted by R², R³, R⁴, R⁵, and R⁶ above may be monosubstituted amino groups or disubstituted amino groups, with disubstituted amino groups being desirable. They desirably have 1 to 10 carbon atoms, preferably 1 to 6 carbon atoms, and more preferably, 1 to 4 carbon atoms. Specific examples are methylamino groups, ethylamino groups, propylamino groups, dimethylamino groups, diethylamino groups, dipropylamino groups, and diphenylamino groups.

The group denoted by R¹, R², R³, R⁴, R⁵, and R⁶ may comprise one or more substituents. Examples of the substituents are alkyl groups, phenyl groups, amino groups, halogen atoms, alkoxy groups, aryloxy groups, alkoxycarbonyl groups, acyloxy groups, acylamino groups, carbamoyl groups, cyano groups, and heterocyclic groups. Of these, alkyl groups are preferred.

In general formula (I), each of A and B independently denotes a substituent, and the combination of A and B is Combinations 1 to 9 below. All of the substituents in the combinations given below are electron-withdrawing substituents. In the compound denoted by general formula (I), as set forth above, having R¹ denote a hydrogen atom or a substituent with a Hammett σp value of equal to or greater than −0.30 and A and B denote electron-withdrawing substituents in one of the combinations given below can achieve absorption characteristics suited to recording in the short wavelength region.

Examples of desirable combinations are Combinations 1, 2, 3, 4, 5, 7, and 8 below. In Combinations 2, 3, and 4, the substituent denoted by A may be identical to or different from the substituent denoted by B.

| | | |
|---|---|---|
| (Combination 1) | A: cyano group, | B: cyano group; |
| (Combination 2) | A: oxycarbonyl group, | B: oxycarbonyl group; |
| (Combination 3) | A: acyl group, | B: acyl group; |
| (Combination 4) | A: sulfonyl group | B: sulfonyl group; |
| (Combination 5) | A: cyano group, | B: acyl group; |
| (Combination 6) | A: cyano group, | B: sulfonyl group; |
| (Combination 7) | A: oxycarbonyl group, | B: acyl group; |
| (Combination 8) | A: oxycarbonyl group, | B: sulfonyl group; |
| (Combination 9) | A: sulfonyl group, | B: acyl group. |

Examples of the above oxycarbonyl group are alkoxycarbonyl groups and aryloxycarbonyl groups. The details thereof are identical to those set forth for the alkoxycarbonyl groups and aryloxycarbonyl groups above. The details of the above acyl group and sulfonyl are also identical to those set forth above.

In the compound denoted by general formula (I), at least one from among R¹, R², R³, R⁴, R⁵, R⁶, A, and B comprises a polymerizable group. In the present invention, the term “polymerizable group” is not specifically limited other than that it be a group capable of forming a polymer by a reaction based on light. By incorporating a polymerizable group, it is possible to record either directly or indirectly an interference fringe in the form of refractive index modulation by irradiating a recording light.

When employing radical polymerization as the recording reaction, examples of the polymerizable group are acryloyl, methacryloyl, acrylamide, methacrylamide, styryl, and vinyl groups. Of these, acryloyl, methacryloyl, acrylamide, and methacrylamide groups are desirable; acryloyl and acrylamide groups are preferred; and acryloyl groups are further preferred. When employing cationic polymerization, examples of the polymerizable group are oxylan, oxetane, propylene carbonate, butyl carbonate, and γ-butyrolactone groups; oxylan and oxetane groups are desirable. It is also possible to apply a cation polymerizable group, but radical polymerizable group is preferred from the perspective of not promoting reactions in a dark place. The substitution position of the polymerizable group is not specifically limited, but is desirably present on at least one or more from among R¹, A, and B, preferably R¹.

In general formula (I), it is desirable for R¹ to denote an alkyl, alkoxy, or acyloxy group; R², R³, R⁴, and R⁵ to denote hydrogen atoms or acyloxy groups; the combination of (A, B) to be Combination 1 (cyano group, cyano group), Combination 2 (oxycarbonyl group, oxycarbonyl group), Combination 3 (acyl group, acyl group), Combination 4 (sulfonyl group, sulfonyl group), Combination 5 (cyano group, acyl group), Combination 7 (oxycarbonyl group, acyl group), or Combination 8 (oxycarbonyl group, sulfonyl group); and for the polymerizable group that is substituted to be a radical polymerizable group. It is preferable for R¹ to denote an alkoxy or acyloxy group; R², R³, R⁴, and R⁵ to denote hydrogen atoms; the combination of (A, B) to be Combination 1 (cyano group, cyano group) or Combination 2 (oxycarbonyl group, oxycarbonyl group); and for the polymerizable group that is substituted to be a radical polymerizable group substituted on R¹.

Specific examples of the compound denoted by general formula (I) are given below. However, the present invention is not limited to these specific examples.

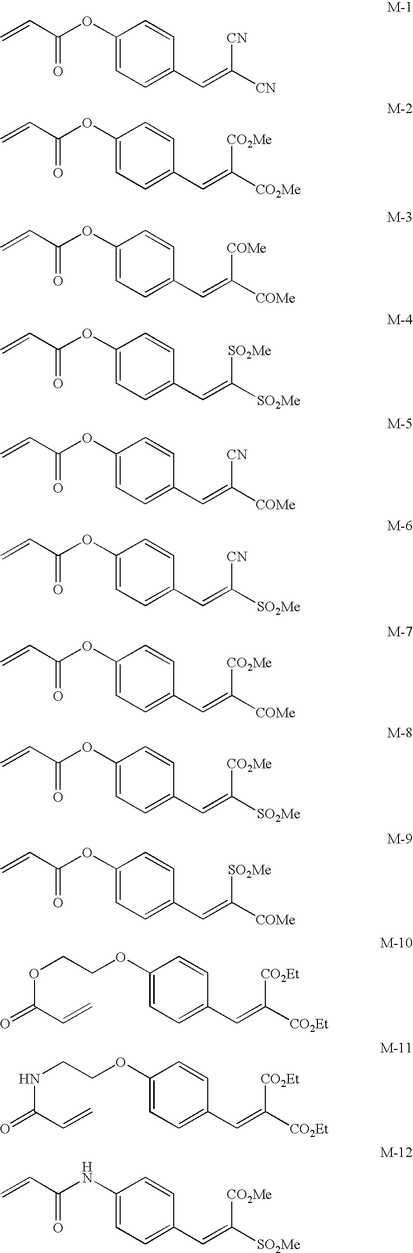

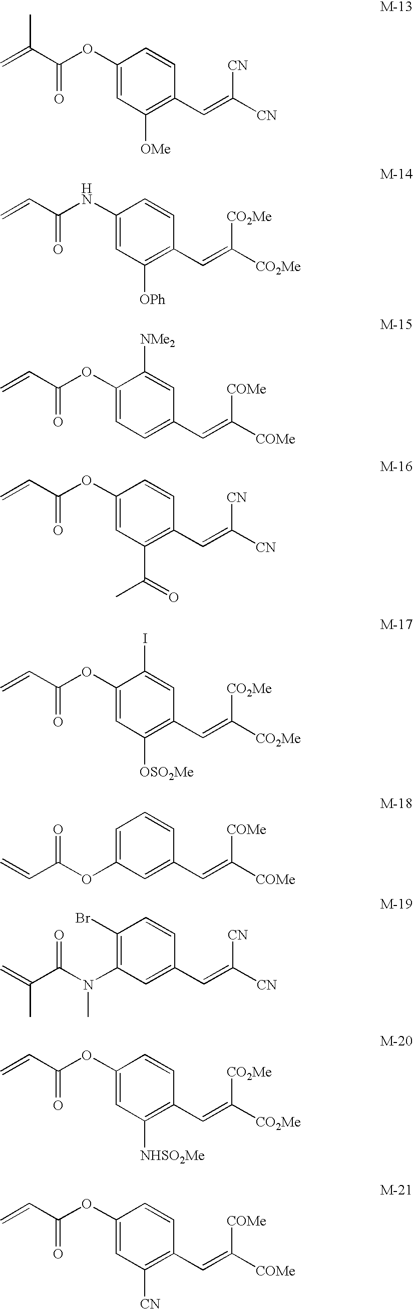

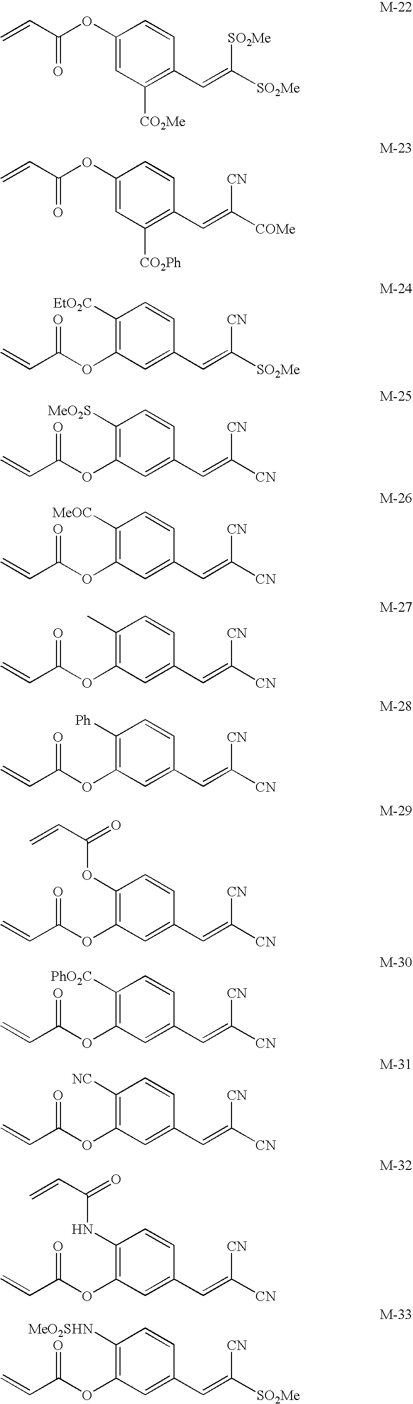

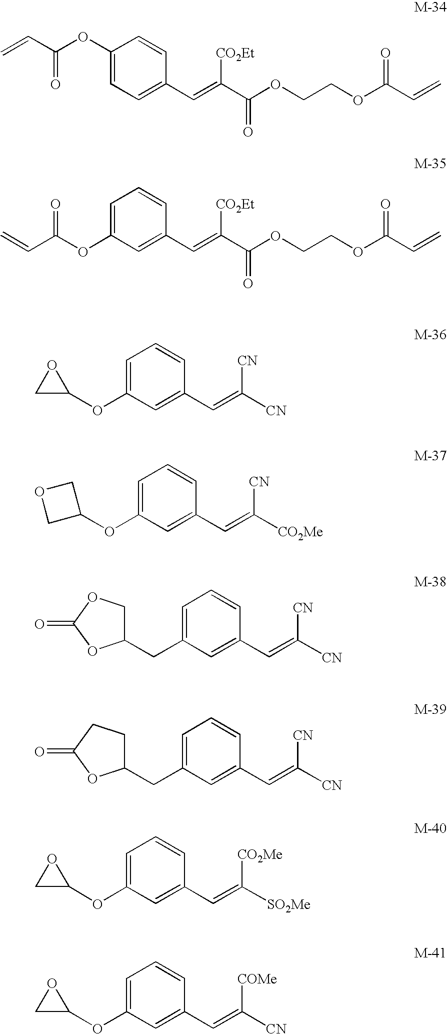

The compound denoted by general formula (I) set forth above can be synthesized by the following scheme, for example. For details of synthesis methods, see the arylidene dye synthesis methods described in Dye Chemistry and Applications (p. 36, Dainippon-tosho Co., by Masaru Matsuoka, 1994, which is expressly incorporated herein by reference in its entirety), and Examples described further below.

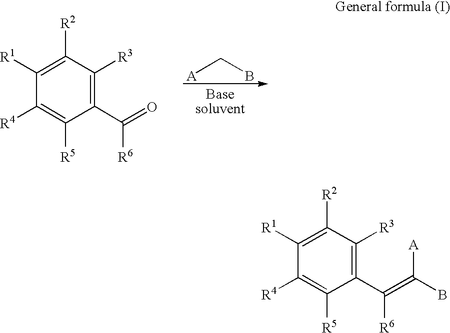

[In the above scheme, R¹ to R⁶, A and B are defined as in general formula (I).]

Absorption at the recording wavelength in the compound employed as the recording compound in a holographic recording medium is desirably low so as to increase medium transmittance and achieve high sensitivity. The compound denoted by general formula (I) above can exhibit a molar absorbance coefficient ε at a wavelength of 405 nm, for example, of equal to or smaller than 200 mol·1 cm⁻¹, and is thus suited to recording at a wavelength of about 400 nm. It is also desirable for achieving high recording capacity for the compound to be great absorption on the side of shorter wavelength than the recording wavelength. The compound denoted by general formula (I) above can have a maximum absorption wavelength λmax of shorter than 405 nm, which is suitable for recording at a wavelength of about 400 nm. Specifically, the molar absorbance coefficient sat ε_{at 405 nm} at a wavelength of 405 nm of the compound denoted by general formula (I) is desirably equal to or smaller than 200 mol·l·cm⁻¹, preferably falling within a range of 0 to 100 mol·l·cm⁻¹. The compound denoted by general formula (I) desirably has a maximum absorption wavelength λmax of shorter than 405 nm, preferably falling within a range of 300 to 350 nm. The molar absorbance coefficient at λmax is desirably equal to or greater than 10,000 mol·l·cm⁻¹, preferably equal to or greater than 30,000 mol·l·cm⁻¹. The upper limit of the molar absorbance coefficient at λmax is not specifically limited. By way of example, it can be about 200,000 mol·l·cm⁻¹.

The above absorption characteristics can be obtained from absorption spectra measured with a spectrophotometer for the ultraviolet and visible regions for a solution obtained by dissolving the compound in a suitable solvent, such as methylene chloride and the like.

The holographic recording composition of the present invention comprises at least the arylidene compound denoted by general formula (I). A single compound denoted by general formula (I) may be employed, or two or more such compounds may be employed in combination. The content of the compound denoted by general formula (I) in the holographic recording composition of the present invention is not specifically limited and may be suitably selected based on the objective. A content of 1 to 50 weight percent is desirable, 1 to 30 weight percent is preferable, and 3 to 10 weight percent is of even greater preference. When the content is equal to or less than 50 weight percent, stability of the interference image can be readily ensured. A content of equal to or more than 1 weight percent can yield properties that are desirable from the perspective of diffraction efficiency.

The compound denoted by general formula (I) may be a monofunctional monomer comprising one polymerizable group per molecule, or may be a multifunctional monomer comprising 2 or more such groups. The holographic recording composition of the present invention may comprise just the compound denoted by general formula (I) as a recording compound, or may comprise other polymerizable monomers in addition to the compound denoted by general formula (I). When employing another polymerizable monomer in combination with the compound denoted by general formula (I), the proportion of the polymerizable monomer employed in combination relative to the total quantity of polymerizable monomer is desirably equal to or less than 50 weight percent.

Examples of other monomers that can be employed in combination are radical polymerizable monomers such as acryloylmorpholine, phenoxyethyl acrylate, isobornyl acrylate, 2-hydroxypropyl acrylate, 2-ethylhexyl acrylate, 1,6-hexanediol diacrylate, tripropylene glycol diacrylate, neopentyl glycol PO-modified diacrylate, 1,9-nonanediol diacrylate, hydroxypivalic acid neopentyl glycol diacrylate, EO-modified bisphenol A diacrylate, polyethylene glycol diacrylate, pentaerythritol triacrylate, pentaerythritol tetraacrylate, pentaerythritol hexaacrylate, EO-modified glycerol triacrylate, trimethylolpropane triacrylate, EO-modified trimethylolpropane triacrylate, 2-naphtho-1-oxyethylacrylate, 2-carbazoyl-9-ylethyl acrylate, (trimethylsilyloxy)dimethylsilylpropyl acrylate, vinyl-1-naphthoate, 2,4,6-tribromophenyl acrylate, pentabromoacrylate, phenylthioethyl acrylate, tetrahydrofurfuryl acrylate, bis-phenoxyethanol fluorene diacrylate, styrene, p-chlorostyrene, N-vinylcarbazol, and N-vinylpyrrolidone. Of these, phenoxyethyl acrylate, 2,4,6-tribromophenyl acrylate, pentabromoacrylate, and bisphenoxyethanol fluorene diacrylate are desirable, and 2,4,6-tribromophenyl acrylate and bisphenoxyethanol fluorene diacrylate are preferred.

Examples of other monomers employed in combination in the form of cationic polymerizable monomers are: 2,3-epoxy-1-propane, 3,4-epoxy-1-butane, 1,6-hexanediol monoglycidyl ether, glycerol diglycidyl ether, glycerol propoxylate diglycidyl ether, glycerol propoxylate diglycidyl ether, glycidyl 4-hydroxyphenyl ether, glycidyl phenyl ether, 1,2-epoxyethylbenzene, bisphenol A diglycidyl ether, pentaerythritol tetra(3-ethyl-3-oxetanylmethyl)ether, 3-ethylene carbonate, propylene carbonate, and γ-butyrolactone.

### Matrix

The recording layer of an optical recording medium normally comprises a polymer to hold the photopolymerization initiator and monomers related to the recording and storage, known as a matrix. The matrix can be employed for achieving enhanced coating properties, coating strength, and hologram recording characteristics. The holographic recording composition of the present invention can comprise curing compounds in the form of a matrix binder and/or matrix forming components (matrix precursors). A method of forming the matrix by, for example, coating a composition containing the matrix precursor on the surface of a substrate and then curing it is desirable because it permits the formation of the recording layer without the use of, or using only a small quantity of, solvent. Thermosetting compounds and light-curing compounds employing catalysts and the like that cure when irradiated with light may be employed as these curing compounds. Thermosetting compounds are desirable from the perspective of recording characteristics.

The thermosetting compound suitable for use in the holographic recording composition of the present invention is not specifically limited. The matrix contained in the recording layer may be suitably selected based on the objective. Examples are urethane resins formed from isocyanate compounds and alcohol compounds; epoxy compounds formed from oxysilane compounds; melamine compounds; formalin compounds; ester compounds of unsaturated acids such as (meth)acrylic acid and itaconic acid; and polymers obtained by polymerizing amide compounds.

Of these, polyurethane matrices formed from isocyanate compounds and alcohol compounds are preferable. From the perspective of recording retention properties, three-dimensional polyurethane matrices formed from polyfunctional isocyanates and polyfunctional alcohols are particularly preferred.

The details of polyfunctional isocyanates and polyfunctional alcohols capable of forming polyurethane matrices are described below bed below.

Examples of the polyfunctional isocyanates are: biscyclohexylmethane diisocyanate, hexamethylene diisocyanate, phenylene-1,3-diisocyanate, phenylene-1,4-diisocyanate, 1-methoxyphenylene-2,4-diisocyanate, 1-methylphenylene-2,4-diisocyanate, 2,4-tolylene diisocyanate, 2,6-tolylene diisocyanate, 1,3-xylylene diisocyanate, 1,4-xylylene diisocyanate, biphenylene-4,4′-diisocyanate, 3,3′-dimethoxybiphenylene-4,4′-diisocyanate, 3,3′-dimethylbiphenylene-4,4′-diisocyanate, diphenylmethane-2,4′-diisocyanate, diphenylmethane-4,4′-diisocyanate, 3,3′-dimethoxydiphenylmethane-4,4′-diisocyanate, 3,3′-dimethyldiphenylmethane-4,4′-diisocyanate, naphthylene-1,5-diisocyanate, cyclobutylene-1,3-diisocyanate, cyclopentylene-1,3-diisocyanate, cyclohexylene-1,3-diisocyanate, cyclohexylene-1,4-diisocyanate, 1-methylcyclohexylene-2,4-diisocyanate, 1-methylcyclohexylene-2,6-diisocyanate, 1-isocyanate-3,3,5-trimethyl-5-isocyanate methylcyclohexane, cyclohexane-1,3-bis(methylisocyanate), cyclohexane-1,4-bis(methylisocyanate), isophorone diisocyanate, dicyclohexylmethane-2,4′-diisocyanate, dicyclohexylmethane-4,4′-diisocyanate, ethylene diisocyanate, tetramethylene-1,4-diisocyanate, hexamethylene-1,6-diisocyanate, dodecamethylene-1,12-diisocyanate, phenyl-1,3,5-triisocyanate, diphenylmethane-2,4,4′-triisocyanate, diphenylmethane-2,5,4′-triisocyanate, triphenylmethane-2,4′,4″-triisocyanate, triphenylmethane-4,4′,4″-triisocyanate, diphenylmethane-2,4,2′,4′-tetraisocyanate, diphenylmethane-2,5,2′,5′-tetraisocyanate, cyclohexane-1,3,5-triisocyanate, cyclohexane-1,3,5-tris(methylisocyanate), 3,5-dimethylcyclohexane-1,3,5-tris(methylisocyanate), 1,3,5-trimethylcyclohexane-1,3,5-tris(methylisocyanate), dicyclohexylmethane-2,4,2′-triisocyanate, dicyclohexylmethane-2,4,4′-triisocyanate lysine isocyanate methyl ester, and prepolymers having isocyanates on both ends obtained by reacting a stoichiometrically excess quantity of one or more of these organic isocyanate compounds with a polyfunctional active hydrogen-containing compound. Of these, biscyclohexylmethane diisocyanate and hexamethylene diisocyanate are preferred. They may be employed singly or in combinations of two or more.

The polyfunctional alcohols may be in the form of a single polyfunctional alcohol, or in the form of a mixture with two or more polyfunctional alcohols. Examples of these polyfunctional alcohols are: glycols such as ethylene glycol, triethylene glycol, diethylene glycol, polyethylene glycol, propylene glycol, polypropylene glycol, and neopentyl glycol; diols such as butanediol, pentanediol, hexanediol, heptanediol, and tetramethylene glycol; bisphenols; compounds in the form of these polyfunctional alcohols modified by polyethyleneoxy chains or polypropyleneoxy chains; and compounds in the form of these polyfunctional alcohols modified by polyethyleneoxy chains or polypropyleneoxy chains, such as glycerin, trimethylolpropane, butanetriol, pentanetriol, hexanetriol, decanetriol, and other triols.

The content of the above-described matrix-forming components (or matrix) in the holographic recording composition of the present invention is desirably 10 to 95 weight percent, preferably 35 to 90 weight percent. When the content is equal to or greater than 10 weight percent, stable interference images can be readily achieved. At equal to or less than 95 weight percent, desirable properties can be obtained from the perspective of diffraction efficiency.

### Photopolymerization Initiator

The holographic recording composition of the present invention can comprise a photopolymerization initiator in addition to the compound denoted by general formula (I). The photopolymerization initiator is not specifically limited other than that it be sensitive to the recording light. Materials inducing a radical polymerization reaction or cationic ring-opening polymerization reaction by light irradiation can be employed as a polymerization initiator. A photo-induced radical polymerization initiator is desirable from the perspective of efficiency of the polymerization reaction.

Examples of such photo-induced radical polymerization initiators are: 2,2′-bis(o-chlorophenyl)-4,4′-5,5′-tetraphenyl-1,1′-biimidazole, 2,4,6-tris(trichloromethyl)-1,3,5-triazine, 2,4-bis(trichloro-methyl)-6-(p-methoxyphenylvinyl)-1,3,5-triazine, diphenyliodoniumtetrafluoroborate, diphenyliodoniumhexafluorophosphate, 4,4′-di-t-butyldiphenyliodoniumtetrafluoroborate, 4-diethylaminophenylbenzenediazoniumhexafluorophosphate, benzoin, 2-hydroxy-2-methyl-1-phenylpropane-2-one, benzophenone, thioxanthone, 2,4,6-trimethylbenzoyl diphenylacyl phosphine oxide, triphenylbutylborate tetraethyl ammonium, diphenyl-4-phenylthiophenyl sulfonium hexafluorophosphate, 2,2-dimethoxy-1,2-diphenylethane-1-one, phenylglyoxylic acid methyl ester, 2-methyl-1-[4-(methylthio)phenyl]-2-morpholinopropane-1-one, bis(2,4,6-trimethylbenzoyl)phenylphosphine oxide, 2,4,6-trimethylbenzoyl diphenyl phosphine oxide, 1,2-octanedione, 1-[4-(phenylthio)-2-(0-benzoyloxime)], and bis(eta 5-2,4-cyclopentadien-1-yl)bis[2,6-difluoro-3-(1H-pyrrol-1-yl)phenyltitanium]. These may be employed singly or in combinations of two or more. A sensitizing dye, described further below, may also be employed in combination based on the wavelength of the light being irradiated.

Among photo-induced radical polymerization initiators, the suitable photo-induced radical polymerization initiator may be a compound denoted by general formula (II).

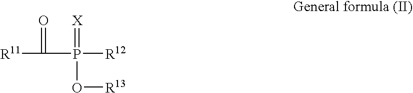

In general formula (II), each of R¹¹, R¹² and R¹³ independently denotes an alkyl group, aryl group or heterocyclic group, and X denotes an oxygen atom or sulfur atom.

The compound denoted by general formula (II) will be described in detail below.

In general formula (II), each of R¹¹, R¹², and R¹³ independently denotes an alkyl group, aryl group, or heterocyclic group.

The alkyl groups denoted by R¹¹, R¹², and R¹³ can be linear or branched, and substituted or unsubstituted. They desirably have 1 to 30 carbon atoms, preferably 1 to 20 carbon atoms.

Examples of the alkyl groups denoted by R¹¹, R¹², and R¹³ are: methyl groups, ethyl groups, normal propyl groups, isopropyl groups, normal butyl groups, isobutyl groups, tertiary butyl groups, pentyl groups, cyclopentyl groups, hexyl groups, cyclohexyl groups, heptyl groups, octyl groups, tertiary octyl groups, 2-ethylhexyl groups, decyl groups, dodecyl groups, octadecyl groups, 2,3-dibromopropyl groups, adamantyl groups, benzyl groups, and 4-bromobenzyl groups. These may be further substituted. Of these, tertiary butyl groups are greatly preferred from the perspective of stability in the presence of nucleophilic compounds, such as water and alcohol.

The aryl groups denoted by R¹¹, R¹², and R¹³ in general formula (II) can be substituted or unsubstituted. They desirably comprise 6 to 30 carbon atoms, preferably 6 to 20 carbon atoms. Specific examples of these aryl groups are: phenyl groups, naphthyl groups, and anthranyl groups. These may be further substituted. Of these, R¹¹ desirably denotes an aryl group in which an alkyl group, aryl group, alkoxy group, or halogen group is present at position 2, and preferably denotes an aryl group in which an alkyl group, aryl group, alkoxy group, or halogen group is present at positions 2 and 6. For example, R¹¹ desirably denotes a 2-methylphenyl group, 2,4,6-trimethylphenyl group, 2,6-dichlorophenyl group, 2,6-dimethoxyphenyl group, or 2,6-trifluoromethylphenyl group, and preferably denotes a 2,4,6-trimethylphenyl group, 2,6-dichlorophenyl group, or 2,6-dimethoxyphenyl group. The presence of the above substituents at position 2, or at positions 2 and 6, is desirable to enhance stability in the presence of nucleophilic compounds, such as water and alcohols, as described in, for example, Jacobi, M., Henne, A. Polymers Paint Colour Journal 1985, 175, 636, which is expressly incorporated herein by reference in its entirety. Details of desirable examples of alkyl groups and aryl groups employed as the above substituents are identical to those set forth for the alkyl groups denoted by R¹¹, R¹², and R¹³ above.

The heterocyclic groups denoted by R¹¹, R¹², and R¹³ in general formula (II) are desirably four to eight-membered rings, preferably four to six-membered rings, and more preferably, five or six-membered rings. Specific examples are: pyridine rings, piperazine rings, thiophene rings, pyrrole rings, imidazole rings, oxazole rings, and thiazole rings. They may be further substituted. Of these hetero rings, pyridine rings are particularly desirable.

When the groups denoted by R¹¹, R¹², and R¹³ in general formula (II) comprise one or more substituents, examples of the substituents are: halogen groups, alkyl groups, alkenyl groups, alkoxy groups, aryloxy groups, alkylthio groups, alkoxycarbonyl groups, aryloxycarbonyl groups, amino groups, acyl groups, alkylaminocarbonyl groups, arylaminocarbonyl groups, sulfonamide groups, cyano groups, carboxy groups, hydroxyl groups, and sulfonic acid groups. Of these, halogen groups, alkoxy groups, and alkylthio groups are particularly desirable. When R¹¹ denotes an aryl group as set forth above, the above substituents are desirably present at position 2, or positions 2 and 6, on the aryl group.

In general formula (II), X denotes an oxygen atom or a sulfur atom, desirably an oxygen atom.

Examples of desirable compounds denoted by general formula (II) are compounds in which R¹¹ denotes an aryl group with an alkyl group, aryl group, alkoxy group, or halogen group present at position 2, R¹² denotes an aryl group, R¹³ denotes an alkyl group, and X denotes an oxygen atom or a sulfur atom. Examples of preferred compounds are compounds in which R¹¹ denotes an aryl group with an alkyl group, aryl group, alkoxy group, or halogen group present at positions 2 and 6, R¹² denotes an aryl group, R¹³ denotes an alkyl group, and X denotes an oxygen atom. Examples of compounds of greater preference are compounds in which R¹¹ denotes a 2,6-dimethoxybenzoyl group or 2,6-dichlorobenzoyl group, R¹² denotes a phenyl group, R¹³ denotes an ethyl group or isopropyl group, and X denotes an oxygen atom.

Specific examples of the phosphorus compound denoted by general formula (II) are given below. However, the present invention is not limited to these specific examples.

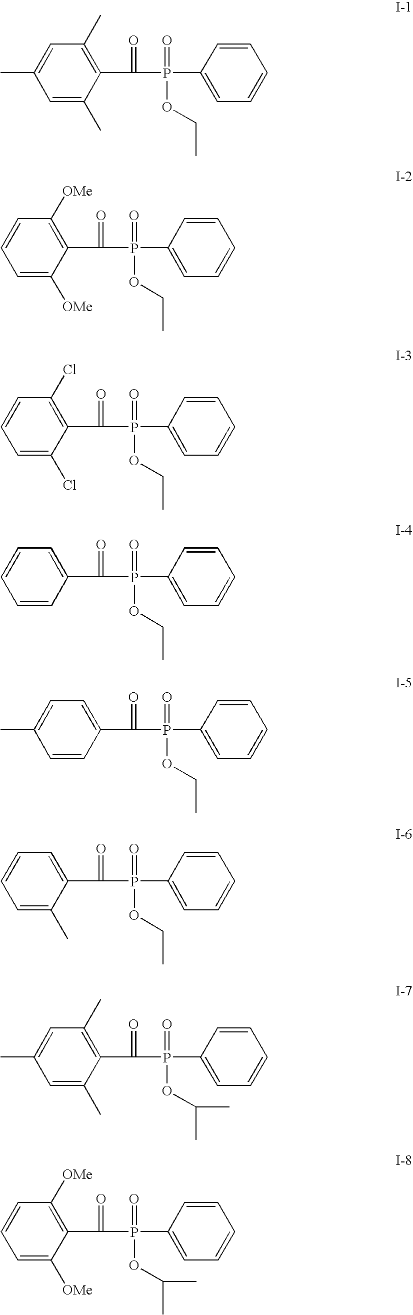

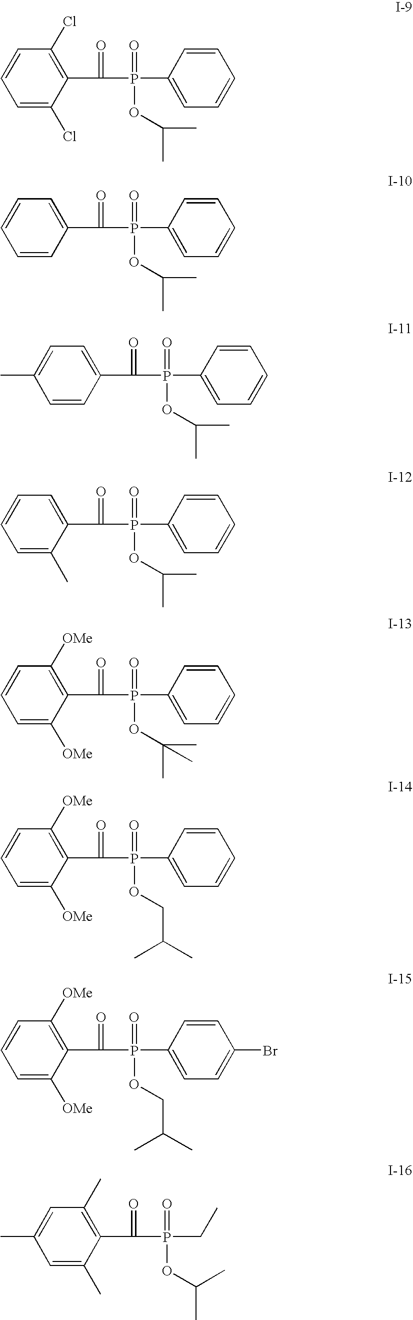

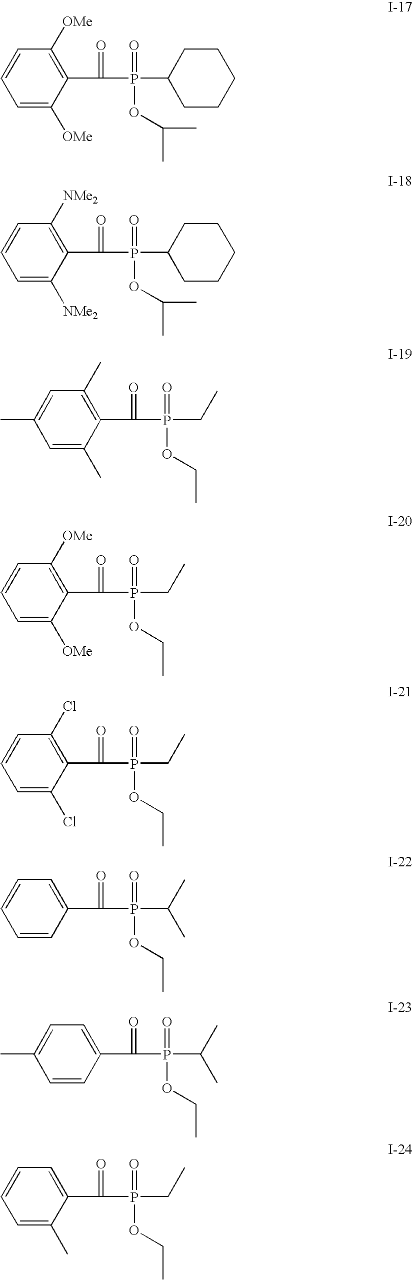

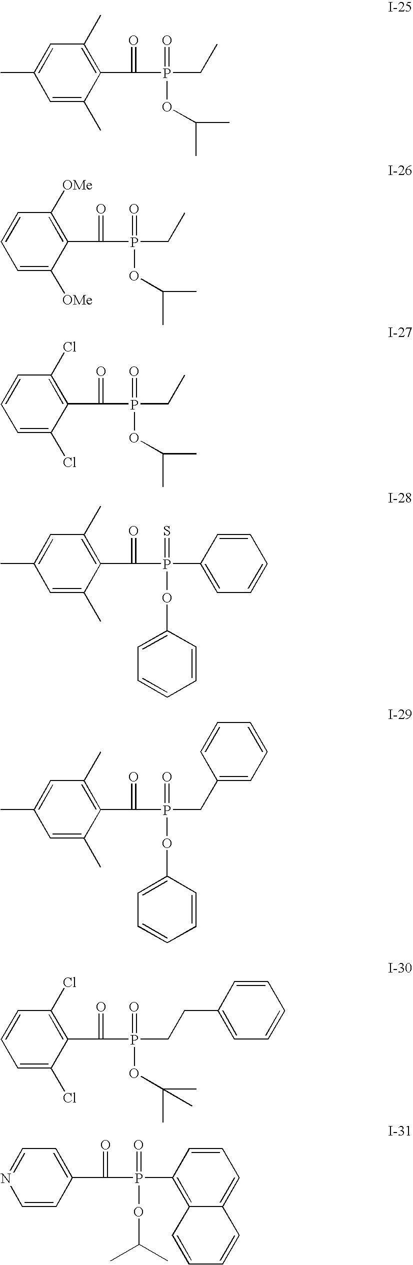

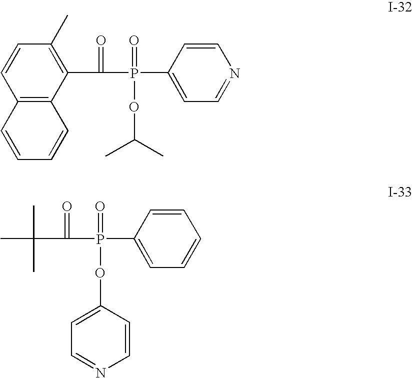

A method of synthesizing the above-described compound denoted by general formula (II) is described in detail in, for example, DE2830927A1, which is expressly incorporated herein by reference in its entirety. Examples described further below can also be referred to for synthesis methods.

Examples of cationic ring-opening photopolymerization initiators are 2,4,6-tris(trichloromethyl)-1,3,5-triazine, 2,4-bis(trichloromethyl)-6-(p-methoxyphenylvinyl)-1,3,5-triazine, diphenyliodonium tetrafluoroborate, 4,4′-di-t-butyldiphenyliodonium tetrafluoroborate, 4-diethylaminophenylbenzenediazonium hexafluorophosphate, and diphenyl-4-phenylthiophenylsulfonium hexafluorophosphate. These may be employed singly or in combinations of two or more. Sensitizing dyes, described further below, may be employed in combination in a manner in conformity with the wavelength of the light that is irradiated.

The content of the photopolymerization initiator in the holographic recording composition of the present invention is desirably 0.01 to 5 weight percent, preferably 1 to 3 weight percent. The content of equal to or greater than 0.01 weight percent can ensure an interference image of good sensitivity. The content of equal to or greater than 5 weight percent can permit the formation of a recording layer having adequate transmittance of the recording light and exhibiting good recording sensitivity.

### Other Components

Polymerization inhibitors and oxidation inhibitors may be added to the holographic recording composition of the present invention to improve the storage stability of the holographic recording composition, as needed.

Examples of polymerization inhibitors and oxidation inhibitors are: hydroquinone, p-benzoquinone, hydroquinone monomethyl ether, 2,6-ditert-butyl-p-cresol, 2,2′-methylenebis(4-methyl-6-tert-butylphenol), triphenylphosphite, trisnonylphenylphoshite, phenothiazine, and N-isopropyl-N′-phenyl-p-phenylenediamine.

The quantity of polymerization inhibitor or oxidation inhibitor added is preferably equal to or less than 3 weight percent of the total quantity of recording monomer. When the quantity added exceeds 3 weight percent, polymerization may slow down, and in extreme cases, ceases.

As needed, a sensitizing dye may be added to the holographic recording composition of the present invention. Known compounds such as those described in “Research Disclosure, Vol. 200, 1980, December, Item 20036” and “Sensitizers” (pp. 160-163, Kodansha, ed. by K. Tokumaru and M. Okawara, 1987) and the like, which are expressly incorporated herein by reference in their entirety, may be employed as sensitizing dyes.

Specific examples of sensitizing dyes are: 3-ketocoumarin compounds described in Japanese Unexamined Patent Publication (KOKAI) Showa No. 58-15603; thiopyrilium salt described in Japanese Unexamined Patent Publication (KOKAI) Showa No. 58-40302; naphthothiazole merocyanine compounds described in Japanese Examined Patent Publications (KOKOKU) Showa Nos. 59-28328 and 60-53300; and merocyanine compounds described in Japanese Examined Patent Publications (KOKOKU) Showa Nos. 61-9621 and 62-3842 and Japanese Unexamined Patent Publications (KOKAI) Showa Nos. 59-89303 and 60-60104, which are expressly incorporated herein by reference in their entirety.

Further examples are the dyes described in “The Chemistry of Functional Dyes” (1981, CMC Press, pp. 393-416) and “Coloring Materials” (60 [4] 212-224 (1987)), which are expressly incorporated herein by reference in their entirety. Specific examples are cationic methine dyes, cationic carbonium dyes, cationic quinoneimine dyes, cationic indoline dyes, and cationic styryl dyes.

Further, keto dyes such as coumarin (including ketocoumarin and sulfonocoumarin) dyes, merostyryl dyes, oxonol dyes, and hemioxonol dyes; nonketo dyes such as nonketo polymethine dyes, triarylmethane dyes, xanthene dyes, anthracene dyes, rhodamine dyes, acrylidine dyes, aniline dyes, and azo dyes; nonketo polymethine dyes such as azomethine dyes, cyanine dyes, carbocyanine dyes, dicarbocyanine dyes, tricarbocyanine dyes, hemicyanine dyes, and styryl dyes; and quinone imine dyes such as azine dyes, oxazine dyes, thiazine dyes, quinoline dyes, and thiazole dyes are included among the spectral sensitizing dyes.

These sensitizing dyes may be employed singly or in combinations of two or more.

A photo-heat converting material can be incorporated into the holographic recording composition of the present invention for enhancing the sensitivity of the recording layer formed with the holographic recording composition.

The photo-heat converting material is not specifically limited, and may be suitably selected based on the functions and properties desired. For example, for convenience during addition to the recording layer with the photopolymer and so as not to scatter incident light, an organic dye or pigment is desirable. From the perspectives of not absorbing and not scattering light from the light source employed in recording, infrared radiation-absorbing dyes are desirable.

Such infrared radiation-absorbing dyes are not specifically limited, and may be suitably selected based on the objective. However, cationic dyes, complex-forming dyes, quinone-based neutral dyes, and the like are suitable. The maximum absorption wavelength of the infrared radiation-absorbing dye preferably falls within a range of 600 to 1,000 nm, more preferably a range of 700 to 900 nm.

The content of infrared radiation-absorbing dye in the holographic recording composition of the present invention can be determined based on the absorbance at the wavelength of maximum absorbance in the infrared region in the recording medium formed with the holographic recording composition of the present invention. This absorbance preferably falls within a range of 0.1 to 2.5, more preferably a range of 0.2 to 2.0.

The holographic recording composition of the present invention can be employed as various holographic recording compositions capable of recording information when irradiated with a light containing information. In particular, it is suited to use as a volume holographic recording composition. A recording layer can be formed by coating the holographic recording composition of the present invention on a substrate, for example. When the holographic recording composition of the present invention contains a thermosetting compound such as those set forth above, a matrix can be formed by promoting the curing reaction by heating following coating. The heating conditions can be determined based on the thermosetting resin employed. The recording layer can be formed by casting when the viscosity of the holographic recording composition is adequately low. When the viscosity is so high that casting is difficult, a dispenser can be employed to spread a recording layer on a lower substrate, and an upper substrate pressed onto the recording layer so as to cover it and spread it over the entire surface, thereby forming a recording medium.

### Holographic Recording Medium

The holographic recording medium of the present invention comprises a recording layer comprising the compound denoted by general formula (I). The recording layer can be formed with the holographic recording composition of the present invention. For example, the recording layer comprised of the holographic recording composition of the present invention can be formed by the above-described method.

The recording layer of the holographic recording medium of the present invention comprises the compound denoted by general formula (I). The compound denoted by general formula (I) can afford absorption characteristics suited to recording by the irradiation of a short-wavelength light, thereby permitting the formation of a holographic recording medium permitting high-density recording with high sensitivity in the short wavelength recording region. The content of the compound denoted by general formula (I) in the recording layer is, as the content in the holographic recording composition of the present invention set forth above, desirably 1 to 50 weight percent, preferably 1 to 30 weight percent, and more preferably, 3 to 10 weight percent. The content not exceeding 50 weight percent readily can ensure interference image stability, and the content of equal to or greater than 1 weight percent can yield desirable properties from the perspective of diffraction efficiency. The details of the various components of the recording layer in the holographic recording medium of the present invention are identical to those set forth above for the holographic recording composition of the present invention.

The holographic recording medium of the present invention is particularly suitable as a holographic recording medium employing a light source with a wavelength of about 400 nm. Since the holographic recording medium employs an entering diffraction light as a signal light, transmittance of the recording and reproducing lights is desirably high. For example, in a recording layer 500 micrometers in thickness, the addition of a polymerizable compound with a molecular weight of 400 in a proportion of 10 weight percent relative to the quantity of a matrix yields a concentration of about 0.018 mol/L. At a recording wavelength of 405 nm, considering the case where an initiator having a molar absorbance coefficient of about 80 mol·l·cm⁻¹ at 405 nm is added in a proportion of 15 molar percent relative to the quantity of polymerizable compound, the transmittance of the recording layer is less than 60 percent when the molar absorbance coefficient of the polymerizable compound is equal to or greater than 200 mol·l·cm⁻¹. Since it is desirable for the transmittance of the recording medium to be equal to or greater than 60 percent, the molar absorbance coefficient of the polymerizable compound is desirably equal to or smaller than 200 mol·l·cm⁻¹. Since the compound denoted by general formula (I) can achieve the above-described desirable absorption characteristics, as set forth above, it is suitably employed as a recording monomer in a holographic recording medium employing a light source with a wavelength of about 400 nm.

The holographic recording medium of the present invention comprises the above recording layer (holographic recording layer), and preferably comprises a lower substrate, a filter layer, a holographic recording layer, and an upper substrate. As needed, it may comprise additional layers such as a reflective layer, filter layer, first gap layer, and second gap layer.

The holographic recording medium of the present invention is capable of recording and reproducing information through utilization of the principle of the hologram. This may be a relatively thin planar hologram that records two-dimensional information or the like, or a volumetric hologram that records large quantities of information, such as three-dimensional images. It may be either of the transmitting or reflecting type. Since the holographic recording medium of the present invention is capable of recording high volumes of information, it is suitable for use as a volume holographic recording medium of which high recording density is demanded.

The method of recording a hologram on the holographic recording medium of the present invention is not specifically limited; examples are amplitude holograms, phase holograms, blazed holograms, and complex amplitude holograms. Among these, a preferred method is the so-called “collinear method” in which recording of information in volume holographic recording regions is carried out by irradiating an informing light and a reference light onto a volume holographic recording area as coaxial beams to record information by means of interference pattern through interference of the informing light and the reference light.

Details of substrates and various layers that can be incorporated into the holographic recording medium of the present invention will be described below.

### —Substrate—

The substrate is not specifically limited in terms of its shape, structure, size, or the like; these may be suitably selected based on the objective. For example, the substrate may be disk-shaped, card-shaped, or the like. A substrate of a material capable of ensuring the mechanical strength of the holographic recording medium can be suitably selected. When the light employed for recording and reproducing enters after passing through the substrate, a substrate that is adequately transparent at the wavelength region of the light employed is desirable.

Normally, glass, ceramic, resin, or the like is employed as the substrate material. From the perspectives of moldability and cost, resin is particularly suitable. Examples of such resins are: polycarbonate resin, acrylic resin, epoxy resin, polystyrene resin, acrylonitrile—styrene copolymers, polyethylene resin, polypropylene resin, silicone resin, fluorine resin, ABS resin, and urethane resin. Of these, from the perspective of moldability, optical characteristics, and cost, polycarbonate resin and acrylic resin are preferred. Synthesized resins and commercially available resins may both be employed as substrates.

Normally, address servo areas are provided on the substrate at prescribed angular intervals as multiple positioning areas extending linearly in a radial direction, with the fan-shaped intervals between adjacent address servo areas serving as data areas. Information for operating focus servos and tracking servos by the sampled servo method, as well as address information, is recorded (preformatted) as pre-embossed pits (servo pits) or the like in address servo areas. Focus servo operation can be conducted using the reflective surface of a reflective film. Wobble pits, for example, can be employed as information for operating a tracking servo. When the holographic recording medium is card-shaped, it is possible not to have a servo pit pattern.

The thickness of the substrate is not specifically limited, and may be suitably selected based on the objective: a thickness of 0.1 to 5 mm is preferable, with 0.3 to 2 mm being preferred. A substrate thickness of equal to or greater than 0.1 mm is capable of preventing shape deformation during disk storage, while a thickness of equal to or less than 5 mm can avoid an overall disk weight generating an excessive load on the drive motor.

### —Recording Layer—

The recording layer can be formed with the holographic recording composition of the present invention and is capable of recording information by holography. The thickness of the recording layer is not specifically limited, and may be suitably selected based on the objective. A recording layer thickness falling within a range of 1 to 1,000 micrometers yields an adequate S/N ratio even when conducting 10 to 300 shift multiplexing, and a thickness falling within a range of 100 to 700 micrometers is advantageous in that it yields a markedly good S/N ratio.

### —Reflective Film—

A reflective film can be formed on the servo pit pattern surface of the substrate.

A material having high reflectance for the informing light and reference light is preferably employed as the material of the reflective film. When the wavelength of the light employed as the informing light and reference light ranges from 400 to 780 nm, examples of desirable materials are Al, Al alloys, Ag, and Ag alloys. When the wavelength of the light employed as the informing light and reference light is equal to or greater than 650 nm, examples of desirable materials are Al, Al alloys, Ag, Ag alloys, Au, Cu alloys, and TiN.

By employing an optical recording medium that reflects light as well as can be recorded and/or erased information such as a DVD (digital video disk) as a reflective film, it is possible to record and rewrite directory information, such as the areas in which holograms have been recorded, when rewriting was conducted, and the areas in which errors are present and for which alternate processing has been conducted, without affecting the hologram.

The method of forming the reflective film is not specifically limited and may be suitably selected based on the objective. Various vapor phase growth methods such as vacuum deposition, sputtering, plasma CVD, optical CVD, ion plating, and electron beam vapor deposition may be employed. Of these, sputtering is superior from the perspectives of mass production, film quality, and the like.

The thickness of the reflective film is preferably equal to or greater than 50 nm, more preferably equal to or greater than 100 nm, to obtain adequate reflectance.

### —Filter Layer—

A filter layer can be provided on the servo pits of the substrate, on the reflective layer, or on the first gap layer, described further below.

The filter layer has a function of reflecting selective wavelengths in which, among multiple light rays, only light of a specific wavelength is selectively reflected, permitting passing one light and reflecting a second light. It also has a function of preventing generation of noise in which irregular reflection of the informing light and the reference light by the reflective film of the recording medium is prevented without a shift in the selectively reflected wavelength even when the angle of incidence varies. Therefore, by stacking filter layers on the recording medium, it is possible to perform optical recording with high resolution and good diffraction efficiency.

The filter layer is not specifically limited and may be suitably selected based on the objective. For example, the filter layer can be comprised of a laminate in which at least one of a dichroic mirror layer, coloring material-containing layer, dielectric vapor deposition layer, single-layer or two- or more layer cholesteric layer and other layers suitably selected as needed is laminated. The thickness of the filter layer is not specifically limited and may be, for example, about 0.5 to 20 micrometers.

The filter layer may be laminated by direct application on the substrate or the like with the recording layer, or may be laminated on a base material such as a film to prepare a filter layer which is then laminated on the substrate.

### —First Gap Layer—

The first gap layer is formed as needed between the filter layer and the reflective film to flatten the surface of the lower substrate. It is also effective for adjusting the size of the hologram that is formed in the recording layer. That is, since the recording layer should form a certain size of the interference region of the recording-use reference light and the informing light, it is effective to provide a gap between the recording layer and the servo pit pattern.

For example, the first gap layer can be formed by applying a material such as an ultraviolet radiation-curing resin from above the servo pit pattern and curing it. When employing a filter layer formed by application on a transparent base material, the transparent base material can serve as the first gap layer.

The thickness of the first gap layer is not specifically limited, and can be suitably selected based on the objective. A thickness of 1 to 200 micrometers is desirable.

### —Second Gap Layer—

The second gap layer is provided as needed between the recording layer and the filter layer.

The material of the second gap layer is not specifically limited, and may be suitably selected based on the objective. Examples are: transparent resin films such as triacetyl cellulose (TAC), polycarbonate (PC), polyethylene terephthalate (PET), polystyrene (PS), polysulfone (PSF), polyvinylalcohol (PVA), and poly(methyl methacrylate) (PMMA); and norbornene resin films such as a product called ARTON film made by JSR Corporation and a product called Zeonoa made by Japan Zeon Co. Of these, those that are highly isotropic are desirable, with TAC, PC, the product called ARTON, and the product called Zeonoa being preferred.

The thickness of the second gap layer is not specifically limited and may be suitably selected based on the objective. A thickness of 1 to 200 micrometers is desirable.

Specific embodiments of the holographic recording medium of the present invention will be described in greater detail below. However, the present invention is not limited to these specific embodiments.

### First Implementation Embodiment

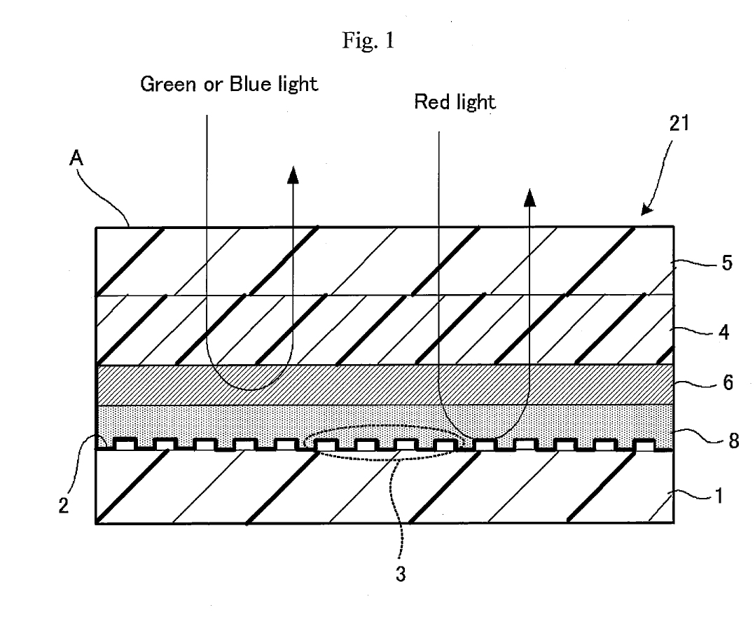

FIG. 1 is a schematic cross-sectional view of the configuration of the holographic recording medium according to the first implementation embodiment. In holographic recording medium 21 according to the first implementation embodiment, a servo pit pattern 3 is formed on substrate 1 made of polycarbonate resin or glass, and aluminum, gold, platinum, or the like is coated on servo pit pattern 3 to provide reflective film 2. In FIG. 1, servo pit pattern 3 has been formed over the entire surface of lower substrate 1, but the servo pit pattern may be formed cyclically. Servo pit pattern 3 is normally 1,750 Angstroms (175 nm) in height, and is quite small relative to the thickness of the substrate and the other layers.

First gap layer 8 is formed by spin coating or the like a material such as an ultraviolet radiation-curing resin on reflective film 2 of lower substrate 1. First gap layer 8 is effective for both the protection of reflective layer 2 and the adjustment of the size of the hologram formed in recording layer 4. That is, providing a gap between recording layer 4 and servo pit pattern 3 is effective for the formation of an interference area for the recording-use reference light and informing light of a certain size in recording layer 4.

Filter layer 6 is provided on first gap layer 8. Recording layer 4 is sandwiched between filter layer 6 and upper substrate 5 (a polycarbonate resin substrate or glass substrate) to form holographic recording medium 21.

FIG. 1 shows a filter layer 6 that passes only infrared radiation and blocks light of all other colors. Accordingly, since the informing light and recording and reproducing-use reference light are blue, they are blocked by filter layer 6 and do not reach reflective film 2. They return, exiting from entry and exit surface A.

Filter layer 6 is a multilayered vapor deposition film comprised of high refractive index layers and low refractive index layers deposited in alternating fashion.

Filter layer 6, comprised of a multilayered vapor deposition film, may be formed directly on first gap layer 8 by vacuum vapor deposition, or a film comprised of a multilayered vapor deposition film formed on a base material may be punched into the shape of a holographic recording medium to employed as filter layer 6.

In this embodiment, holographic recording medium 21 may be disk-shaped or card-shaped. When card-shaped, the servo pit pattern may be absent. In holographic recording medium 21, the lower substrate is 0.6 mm, first gap layer 8 is 100 micrometers, filter layer 6 is 2 to 3 micrometers, recording layer 4 is 0.6 mm, and upper substrate 5 is 0.6 mm in thickness, for a total thickness of about 1.9 mm.

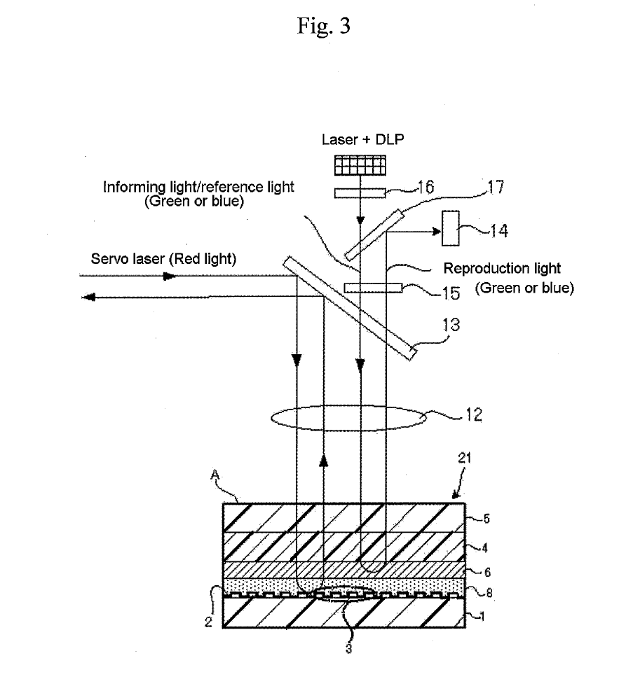

An optical system applicable for the recording of information on and the reproduction of information from holographic recording medium 21 will be described with reference to FIG. 3.

First, a light (red light) emitted by a servo laser is nearly 100 percent reflected by dichroic mirror 13, passing through objective lens 12. Objective lens 12 directs the servo light onto holographic recording medium 21 so that it focuses at a point on reflective film 2. That is, dichroic mirror 13 passes light of green and blue wavelengths while reflecting nearly 100 percent of red light. The servo light entering entry and exit surface A to which and from which the light enters and exits of holographic recording medium 21 passes through upper substrate 5, recording layer 4, filter layer 6, and first gap layer 8, is reflected by reflective layer 2, and passes back through first gap layer 8, filter layer 6, recording layer 4, and upper substrate 5, exiting entry and exit surface A. The returning light that exits passes through objective lens 12, is nearly 100 percent reflected by dichroic mirror 13, and the servo information is detected by a servo information detector (not shown in FIG. 3). The servo information that is detected is employed for focus servo, tracking servo, slide servo, and the like. When the hologram material included in recording layer 4 is not sensitive to red light, the servo light passes through recording layer 4 without affecting recording layer 4, even when the servo light is randomly reflected by reflective film 2. Since the light in the form of the servo light reflected by reflective film 2 is nearly 100 percent reflected by dichroic mirror 13, the servo light is not detected by a CMOS sensor or CCD 14 for reproduction image detection and thus does not constitute noise to the reproduction light.

The informing light and recording-use reference light generated by the recording/reproducing laser passes through polarizing plate 16 and is linearly polarized. It then passes through half mirror 17, becoming circularly polarized light at the point where it passes through ¼ wavelength plate 15. The light then passes through dichroic mirror 13, and is directed by objective lens 12 onto holographic recording medium 21 so that the informing light and recording-use reference light form an interference pattern in recording layer 4. The informing light and recording-use reference light enter through entry and exit surface A, interfering with each other to form an interference pattern in recording layer 4. Subsequently, the informing light and recording-use reference light pass through recording layer 4, entering filter layer 6. However, they are reflected before reaching the bottom surface of filter layer 6, returning. That is, neither the informing light nor the recording-use reference light reaches reflective film 2. That is because filter layer 6 is a multilayered vapor deposition layer in which multiple high refractive index and low refractive index layers are alternatively laminated, and has the property of passing only red light.

### Second Implementation Embodiment

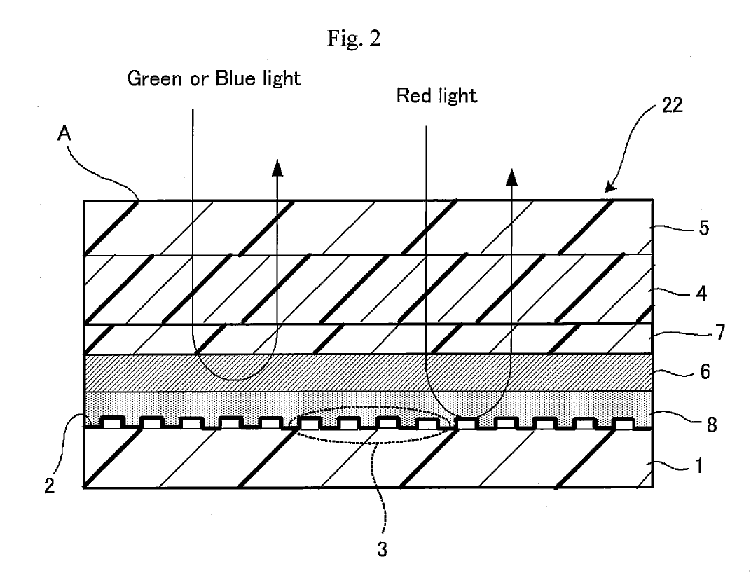

FIG. 2 is a schematic cross-sectional view of the configuration of the holographic recording medium according to the second implementation embodiment. A servo pit pattern 3 is formed on substrate 1 made of polycarbonate resin or glass in the holographic recording medium 22 according to the second implementation embodiment. Reflective film 2 is provided by coating aluminum, gold, platinum, or the like on the surface of servo pit pattern 3. Servo pit pattern 3 is normally 1,750 Angstroms (175 nm) in height in the same manner as in the first implementation embodiment.

The configuration of the second implementation embodiment differs from that of the first implementation embodiment in that second gap layer 7 is provided between filter layer 6 and recording layer 4 in holographic recording medium 22 according to the second implementation embodiment. A point at which the informing light and reproduction light are focused is present in second gap layer 7. When this area is embedded in a photopolymer, excessive consumption of monomer occurs due to excess exposure, and multiplexing recording capability diminishes. Accordingly, it is effective to provide a nonreactive transparent second gap layer.

Filter layer 6 in the form of a multilayered vapor deposition film comprised of multiple layers in which multiple high refractive index and low refractive index layers are alternately laminated is formed over first gap layer 8 once first gap layer 8 has been formed, and the same one as employed in the first implementation embodiment can be employed as filter layer 6 in the second implementation embodiment.

In holographic recording medium 22 of the second implementation embodiment, lower substrate 1 is 1.0 mm, first gap 8 is 100 micrometers, filter layer 6 is 3 to 5 micrometers, second gap layer 7 is 70 micrometers, recording layer 4 is 0.6 mm, and upper substrate 5 is 0.4 mm in thickness, for a total thickness of about 2.2 mm.

When recording or reproducing information, a red servo light and a green informing light and recording/reproducing reference light are directed onto holographic recording medium 22 of the second implementation embodiment having the configuration set forth above. The servo light enters through entry and exit surface A, passing through recording layer 4, second gap layer 7, filter layer 6, and first gap layer 8, and is reflected by reflective film 2, returning. The returning light then passes sequentially back through first gap layer 8, filter layer 6, second gap layer 7, recording layer 4, and upper substrate 5, exiting through entry and exit surface A. The returning light that exits is used for focus servo, tracking servo, and the like. When the hologram material included in recording layer 4 is not sensitive to red light, the servo light passes through recording layer 4 and is randomly reflected by reflective film 2 without affecting recording layer 4. The green informing light and the like enters through entry and exit surface A, passing through recording layer 4 and second gap layer 7, and is reflected by filter layer 6, returning. The returning light then passes sequentially back through second gap layer 7, recording layer 4, and upper substrate 5, exiting through entry and exit layer A. During reproduction, as well, the reproduction-use reference light and the reproduction light generated by irradiating the reproduction-use reference light onto recording layer 4 exit through entry and exit surface A without reaching reflective film 2. The optical action around holographic recording medium 22 (objective lens 12, filter layer 6, and detectors in the form of CMOS sensors or CCD 14 in FIG. 3) is identical to that in the first implementation embodiment and thus the description thereof is omitted.

The method of recording information on the holographic recording medium of the present invention will be described below.

An interference image can be formed on the recording layer of the holographic recording medium of the present invention by irradiation of an informing light and a reference light to the recording layer, and a fixing light can be irradiated to the recording layer on which the interference image has been formed to fix the interference image.

A light having coherent properties can be employed as the informing light. By irradiating the informing light and reference light onto the recording medium so that the optical axes of the informing light and reference light are coaxial, it is possible to record in the recording layer an interference image generated by interference of the informing light and reference light. Specifically, a informing light imparted with a two dimensional intensity distribution and a reference light of intensity nearly identical to that of the informing light are superposed in the recording layer and the interference pattern that they form is used to generate an optical characteristic distribution in the recording layer, thereby recording information. The wavelengths of the informing light and reference light are preferably equal to or greater than 400 nm, more preferably 400 to 2,000 nm, further preferably 400 to 700 nm, and particularly preferably, 405 nm.

After recording information (forming an interference image) by irradiating the informing light and reference light, a fixing light can be irradiated to fix the interference image. The wavelength of the fixing light is preferably less than 400 nm, more preferably equal to or greater than 100 nm but less than 400 nm, and further preferably, equal to or greater than 200 nm but less than 400 nm.

Information can be reproduced by irradiating a reference light onto an interference image formed by the above-described method. In the course of reading (reproducing) information that has been written, just a reference light is irradiated onto the recording layer with the same arrangement as during recording, causing a reproduction light having an intensity distribution corresponding to the optical characteristic distribution formed in the recording layer to exit the recording layer.

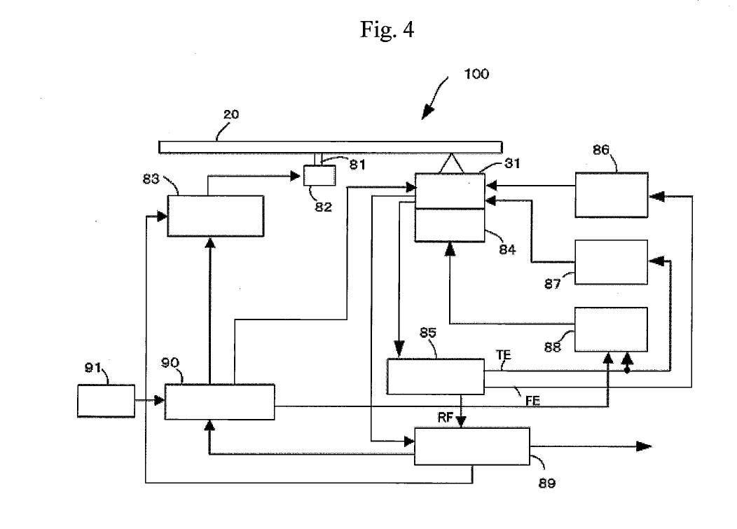

An optical recording and reproducing device suited to use in the recording and reproducing of information in the holographic recording medium of the present invention will be described with reference to FIG. 4.

The optical recording and reproducing device 100 of FIG. 4 is equipped with spindle 81 on which is mounted holographic recording medium 20, spindle motor 82 rotating spindle 81, and spindle servo circuit 83 controlling spindle motor 82 so that it maintains holographic recording medium 20 at a prescribed rpm level.

Recording and reproducing device 100 is further equipped with pickup 31 for recording information by irradiating a informing light and a recording-use reference light onto holographic recording medium 20, and for reproducing information that has been recorded on holographic recording medium 20 by irradiating a reproducing-use reference light onto holographic recording medium 20 and detecting the reproduction light; and driving device 84 capable of moving pickup 31 radially with respect to holographic recording medium 20.

Optical recording and reproducing device 100 is equipped with detection circuit 85 for detecting focus error signal FE, tracking error signal TE, and reproduction signal RF based on the output signals of pickup 31; focus servo circuit 86 that operates a focus servo by driving an actuator in pickup 31 to move an objective lens (not shown in FIG. 4) in the direction of thickness of holographic recording medium 20 based on focus error signal FE detected by detection circuit 85; tracking servo circuit 87 that operates a tracking servo by driving an actuator in pickup 31 to move an objective lens in the radial direction of holographic recording medium 20 based on tracking error signal TE detected by detection circuit 85; and slide servo circuit 88 that operates a slide servo by controlling drive device 84 to move pickup 31 in the radial direction of holographic recording medium 20 based on instructions from a controller, described further below, and tracking error signal TE.

Optical recording and reproducing device 100 is further equipped with signal processing circuit 89 that decodes the output data of a CCD array or CMOS in pickup 31 to reproduce data recorded in the data areas of holographic recording medium 20, reproduces a base clock based on reproduction signal RF from detection circuit 85, and determines addresses; controller 90 that effects overall control of optical recording and reproducing device 100; and operation element 91 providing various instructions to controller 90. Controller 90 inputs the base clock and address information outputted by signal processing circuit 89 and controls pickup 31, spindle servo circuit 83, slide servo circuit 88, and the like. Spindle servo circuit 83 inputs the base clock that is outputted by signal processing circuit 89. Controller 90 comprises a central processing unit (CPU), read only memory (ROM), and random access memory (RAM). The functions of controller 90 can be realized by having the CPU that employs the RAM as a work area and execute programs stored in the ROM.

### EXAMPLES

The present invention will be described in detail below based on examples. However, the present invention is not limited to the examples.

### Synthesis Example 1

### Synthesis of Example Compound (M-1)

Example Compound (M-1) was synthesized by the following scheme. The identification results are given below.

¹H NMR (300 MHz, CDCl₃) δ6.11 (d, 1H), 6.32 (dd, 1H), 6.64 (d, 1H), 7.36 (d, 2H), 7.78 (s, 1H), 7.94 (d, 2H). λmax=321 nm(ε=26900) in CH₂Cl₂, ε_{at 405 nm}=131.

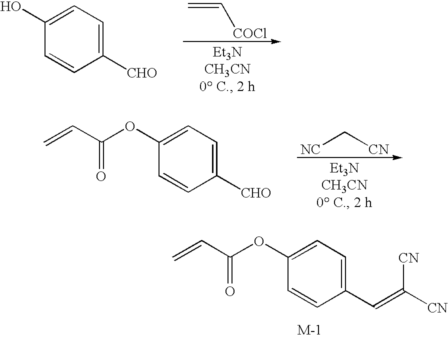

### Synthesis Example 2

### Synthesis of Example Compound (M-10)

Example Compound (M-10) was synthesized by the following scheme. The identification results are given below.

¹H NMR (300 MHz, CDCl₃) δ1.32 (t, 6H), 4.21-4.40 (m, 6H), 4.52 (dd, 2H), 5.89 (d, 1H), 6.15 (dd, 1H), 6.48 (d, 1H), 6.88 (d, 2H), 7.43 (d, 2H), 7.72 (s, 1H). λmax=311 nm(ε=24900) in CH₂Cl₂, ε_{at 405 nm}=20.

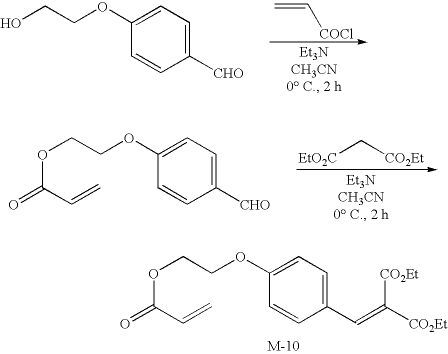

### Synthesis Example 3

### Synthesis Example of Compounds Denoted by General Formula (II)

Example Compounds (I-2), (I-3), (I-8), and (I-9) were synthesized by the general scheme given below based on the method described in DE2830927A1. In the following scheme, R¹¹ to R¹³ have the same definitions as in general formula (II). Various compounds in which R¹¹ to R¹³ vary can be synthesized by the following scheme by employing different starting materials in synthesis.

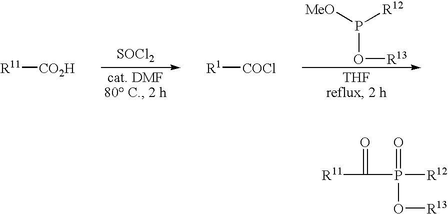

The identification results of Example Compounds (I-2), (I-3), (I-8) and (I-9) thus obtained are given below. <I-2>

¹H NMR (300 MHz, CDCl₃) δ1.32 (t, 3H), 3.62(s, 6H), 4.13-4.26 (m, 2H), 6.49 (d, 2H), 7.32(t, 1H), 7.40-7.51 (m, 2H), 7.54-7.59(m, 1H), 7.79 (dd, 2H) <I-3>

¹H NMR (300 MHz, CDCl₃) δ1.37 (d, 3H), 1.39 (d, 3H), 4.91-4.98 (m, 1H), 7.29 (s, 3H), 7.47-7.51 (m, 2H), 7.59-7.61 (m, 1H), 7.91 (dd, 2H) <I-8>

¹H NMR (300 MHz, CDCl₃) δ1.34 (d, 3H), 1.38 (d, 3H), 3.67(s, 6H), 4.68-4.80 (m, 1H), 7.32 (t, 1H), 7.41-7.50 (m, 2H), 7.52-7.59 (m, 1H), 7.90 (dd, 2H) <I-9>

¹H NMR (300 MHz, CDCl₃) δ1.36 (t, 3H), 4.41 (q, 2H), 7.28 (s, 3H), 7.58-7.64 (m, 1H), 7.93 (dd, 2H)

### Example 1

### Preparation of Holographic Recording Composition

A 6.4 g quantity of hexamethylene diisocyanate (made by Mitsui Chemicals Polyurethanes, Inc.; trade name: Takenate T-700), 5.21 g of polypropylene oxide triol (made by Mitsui Chemicals Polyurethanes, Inc.; trade name: MN-300), 4.64 g of polyethylene glycol (made by Tokyo Chemical Industry Co., Ltd.), 1.85 g of Example Compound (M-1), 0.16 g of photopolymerization initiator (2,4,6-trimethylbenzoyl-phenylphosphinic acid ethyl ester; trade name: Lucirin TPO-L, made by BASF Japan), and 0.20 g of amine curing catalyst (made by SAN-APRO; trade name: U-CAT 410) were mixed under a nitrogen gas flow to prepare a holographic recording composition.

### Example 2

### Preparation of Holographic Recording Composition

With the exception that the 1.85 g of Example Compound (M-1) in Example 1 was replaced with 1.85 g of Example Compound (M-10), a holographic recording composition was prepared in the same manner as in Example 1.

### Example 3

### Preparation of Holographic Recording Composition

With the exception that the 0.16 g of photopolymerization initiator (2,4,6-trimethylbenzoylphenylphosphinic acid ethyl ester; trade name: Lucirin TPO-L, made by BASF Japan) in Example 1 was replaced with 0.16 g of Example Compound (I-8), a holographic recording composition was prepared in the same manner as in Example 1.

### Example 4

### Preparation of Holographic Recording Composition

With the exception that the 0.16 g of photopolymerization initiator (2,4,6-trimethylbenzoylphenylphosphinic acid ethyl ester; trade name: Lucirin TPO-L, made by BASF Japan) in Example 2 was replaced with 0.16 g of Example Compound (I-8), a holographic recording composition was prepared in the same manner as in Example 2.

### Comparative Example 1

### Preparation of Holographic Recording Composition

A 6.4 g quantity of hexamethylene diisocyanate (made by Mitsui Chemicals Polyurethanes, Inc.; trade name: Takenate T-700), 5.21 g of polypropylene oxide triol (made by Mitsui Chemicals Polyurethanes, Inc.; trade name: MN-300), 4.64 g of polyethylene glycol (made by Tokyo Chemical Industry Co., Ltd.), 1.85 g of 2,4,6-tribromophenyl acrylate (Dai-ichi Kogyo Seiyaku Co., Ltd.; trade name BR-30), 0.16 g of photopolymerization initiator (2,4,6-trimethylbenzoylphenylphosphinic acid ethyl ester; trade name: Lucirin TPO-L, made by BASF Japan), and 0.20 g of amine curing catalyst (made by SAN-APRO; trade name: U-CAT 410) were mixed under a nitrogen gas flow to prepare a holographic recording composition.

### Comparative Example 2

### Preparation of Holographic Recording Composition

With the exception that the 1.85 g of 2,4,6-tribromophenyl acrylate (Dai-ichi Kogyo Seiyaku Co., Ltd.; trade name BR-30) employed in Comparative Example 1 was replaced with 1.85 g of the following monomer (R-1) (λmax=435 nm (ε=58600) in CH₂Cl₂, ε_{at 405 nm}=26,758) described in Japanese Unexamined Patent Publication (KOKAI) No. 2005-275158, a holographic recording composition was prepared in the same manner as in Comparative Example 1.

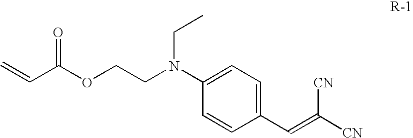

### Comparative Example 3

### Preparation of Holographic Recording Composition

With the exception that the 1.85 g of 2,4,6-tribromophenyl acrylate (Dai-ichi Kogyo Seiyaku Co., Ltd.; trade name BR-30) employed in Comparative Example 1 was replaced with 1.85 g of the following monomer (R-2) (λmax=460 nm (ε=68,000) in CH₂Cl₂, ε_{at 405 nm}=45,200) described in Japanese Unexamined Patent Publication (KOKAI) No. 2007-272044, a holographic recording composition was prepared in the same manner as in Comparative Example 1.

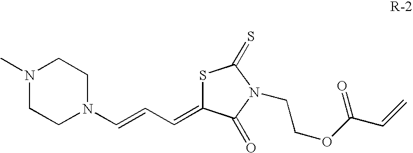

### Examples 5 to 8 and Comparative Examples 4 to 6

### Preparation of Optical Recording Media

A first substrate was prepared by subjecting one side of a glass sheet 0.5 mm in thickness to an antireflective treatment to impart a reflectance of 0.1 percent for perpendicularly incident light with the wavelength of 405 nm.

A second substrate was prepared by subjecting one side of a glass sheet 0.5 mm in thickness to an aluminum vapor deposition treatment to impart a reflectance of 90 percent for perpendicularly incident light with the wavelength of 405 nm.

A transparent polyethylene terephthalate sheet 500 micrometers in thickness was provided as a spacer on the side of the first substrate that had not been subjected to the antireflective treatment.

The holographic recording compositions of Examples 1 to 4 and Comparative Examples 1 to 3 were each separately placed on first substrates, the aluminum vapor deposited surface of the second substrates were stacked on the holographic recording composition in such a manner that air was not entrained, and the first and second substrates were bonded through the spacer. Subsequently, Examples 5 to 8 and Comparative Examples 4 to 6 were left for 6 hours at 80° C. to prepare various optical recording media (holographic recording media). The thickness of the recording layers formed was 200 micrometers in all media prepared.

### <Recording in the Optical Recording Medium and Evaluation>

(1) Measurement of Recording Sensitivity

Employing a hologram recording and reproduction tester, a series of multiplexed holograms was written into the various optical recording media that had been prepared at a spot recording diameter of 200 micrometers at the focal position of the recording hologram, and the sensitivity (recording energy) was measured as follows.

### —Sensitivity Measurement—

The beam energy during recording (mJ/cm²) was varied and the change in the error rate (BER: bit error rate) of the reproduced signal was measured. Normally, there is such a tendency that the luminance of the reproduced signal increases and the BER of the reproduced signal gradually drops with an increase in the irradiated light energy. In the measurement, the lowest light energy at which a fairly good reproduced image (BER<10⁻³) was obtained was adopted as the recording sensitivity of the holographic recording medium. The wavelength of the informing light and reference light for recording as well as the wavelength of the reproduction light were 405 nm.

(2) Measurement of Recording Capacity by Planar Wave Tester

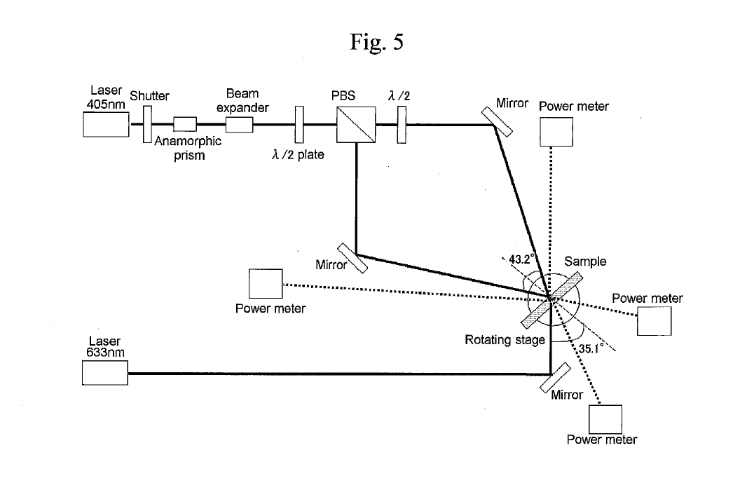

FIG. 5 shows a schematic of the optical system of a planar wave recording tester. A “Littrow” blue laser made by SONY (wavelength: 405 nm) was employed as the recording light source and an He—Ne laser (wavelength: 633 nm) that was not absorbed by the medium was employed as the probe light source. The luminous energy of the recording light source was 4 [mW] with the informing light and reference light combined. The luminous energy of the probe light source was 5 [mW]. The crossing angle of the informing light and the reference light was 43.2° (grating interval: 550 nm). The angle of incidence of the probe light—the angle at which the Bragg condition was satisfied—was 35.1°. A recording spot diameter of 6 mm was employed. The dynamic range of the storage capacity is denoted by an index referred to as “M#”. The recording capacity of each of the optical recording media of Example 5 to 8 and Comparative Examples 4 to 6 was measured with the above-described optical system. The measurement is described below.

Adopting a diffraction efficiency of 1 to 3 percent per cycle as standard, in a manner not exceeding 10 percent, 61 multiplexed recordings were conducted at intervals of 1° from −30° to +30° until the sensitivity of the recording material almost disappeared. Fixing was conducted until absorption of the recording light source by the sample almost ceased (fixing light source: High-power UV-LED (UV-400) made by Keyence), the angular selectivity was evaluated at 0.01° intervals from −32° to +32°, and the square roots of the diffraction efficiencies ηᵢ of the peaks obtained were summed to calculate M#. Diffraction efficiency η was evaluated as set forth below. The results are given in Table 1.

η=diffracted light/(diffracted light+transmitted light)×100

M#=Σ√ηᵢ

(3) Measurement of Transmittance T

The transmittance at a wavelength of 405 nm was measured with a UV-3600 (made by Shimadzu Corporation) for each of the optical recording media prepared in Examples 5 to 8 and Comparative Examples 4 to 6.

(4) Molar Absorbance Coefficient

Each of the recording monomers contained in the holographic recording compositions prepared in Examples 1 to 4 and Comparative Examples 1 to 3 was dissolved in methylene chloride to a concentration of 5×10⁻⁵ mol/L, the absorption spectrum of each solution prepared was measured with a UV-3600 (made by Shimadzu Corporation), and the absorption at 405 nm was measured. The molar absorbance coefficient was calculated from the absorbance thus measured. The maximum absorption wavelength λmax and the molar absorbance coefficient at λmax were also obtained from the absorption spectra. The results are given in Table 1.

TABLE 1

| | Holographic | Recording | Recording | Transmittance T(%) | ε | | ε |
|---|---|---|---|---|---|---|---|
| | recording | sensitivity | capacity | of the medium | at 405 nm | λmax | at λmax |
| | composition | (mJ/cm²) | (M#) | at 405 nm | (mol · l · cm⁻¹) | (nm) | (mol · l · cm⁻¹) |
| Ex. 5 | Ex. 1 | 43 | 12.7 | 65.0 | 131 | 321 | 26900 |
| Ex. 6 | Ex. 2 | 50 | 12.1 | 82.0 | 20 | 311 | 24900 |
| Ex. 7 | Ex. 3 | 39 | 13.2 | 68.3 | 131 | 321 | 26900 |
| Ex. 8 | Ex. 4 | 43 | 12.8 | 86.0 | 20 | 311 | 24900 |
| Comp. | Comp. | 80 | 9.0 | 85.3 | 0 | — | — |
| Ex. 4 | Ex. 1 | | | | | | |
| Comp. | Comp. | — | — | 0.0 | 26700 | 435 | 58600 |
| Ex. 5 | Ex. 2 | | | | | | |
| Comp. | Comp. | — | — | 0.0 | 45200 | 460 | 68000 |
| Ex. 6 | Ex. 3 | | | | | | |

The results of Table 1 show that the optical recording media of Examples 5 to 8, in which the holographic recording compositions of Examples 1 to 4 were employed, all had better recording sensitivity and greater recording capacity than the optical recording media of Comparative Examples 4 to 6, in which the holographic recording compositions of Comparative Examples 1 to 3 were employed.

Recording and reproduction were impossible with Comparative Examples 5 and 6.

The holographic recording composition of the present invention is capable of high density recording, and is thus suitable for use in the manufacturing of various volume hologram-type optical recording media capable of high-density image recording.

Although the present invention has been described in considerable detail with regard to certain versions thereof, other versions are possible, and alterations, permutations and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

What is claimed is:

1. A holographic recording composition comprising a compound denoted by general formula (I). 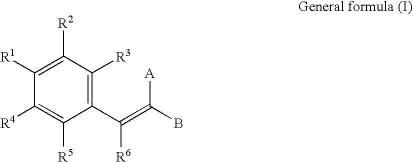
In general formula (I), R¹ denotes a hydrogen atom or a substituent having a Hammett value, σp, of equal to or greater than −0.30, each of R², R³, R⁴, R⁵, and R⁶ independently denotes a hydrogen atom or a substituent, each of A and B independently denotes a substituent and a combination of A and B is one of Combinations 1 to 9, and at least one of R¹, R², R³, R⁴, R⁵, R⁶, A, and B comprises a polymerizable group.

| | | |
|---|---|---|
| (Combination 1) | A: cyano group, | B: cyano group; |
| (Combination 2) | A: oxycarbonyl group, | B: oxycarbonyl group; |
| (Combination 3) | A: acyl group, | B: acyl group; |
| (Combination 4) | A: sulfonyl group, | B: sulfonyl group; |
| (Combination 5) | A: cyano group, | B: acyl group; |
| (Combination 6) | A: cyano group, | B: sulfonyl group; |
| (Combination 7) | A: oxycarbonyl group, | B: acyl group; |
| (Combination 8) | A: oxycarbonyl group, | B: sulfonyl group; |
| (Combination 9) | A: sulfonyl group, | B: acyl group. |

2. The holographic recording composition according to claim 1, wherein, in general formula (I), R¹ denotes a hydrogen atom, alkyl group, aryl group, heterocyclic group, acyl group, sulfonyl group, acyloxy group, alkoxycarbonyl group, aryloxycarbonyl group, alkoxy group, aryloxy group, acylamino group, sulfonylamino group, cyano group, or halogen group, each of R², R³, R⁴, and R⁵ independently denotes a hydrogen atom, alkyl group, aryl group, heterocyclic group, acyl group, sulfonyl group, acyloxy group, alkoxycarbonyl group, aryloxycarbonyl group, alkoxy group, aryloxy group, amino group, acylamino group, sulfonylamino group, or halogen group, and R⁶ denotes a hydrogen atom or an alkyl group.

3. The holographic recording composition according to claim 1, wherein, in general formula (I), R¹ denotes an alkyl group, aryl group, alkoxycarbonyl group, acyloxy group, or halogen group.

4. The holographic recording composition according to claim 1, wherein, in general formula (I), each of R², R³, R⁴, and R⁵ independently denotes a hydrogen atom, alkyl group, aryl group, alkoxy group, aryloxy group, amino group, acyl group, acyloxy group, or halogen group.

5. The holographic recording composition according to claim 1, wherein, in general formula (I), the combination of A and B is Combination 1, 2, 3, 4, 5, 7, or 8.

6. The holographic recording composition according to claim 1, wherein the polymerizable group is a radical polymerizable group.

7. The holographic recording composition according to claim 1, wherein the compound denoted by general formula (I) has a molar absorbance coefficient of equal to or smaller than 200 mol·l·cm⁻¹ at a wavelength of 405 nm.

8. The holographic recording composition according to claim 1, wherein the compound denoted by general formula (I) has a maximum absorption wavelength of shorter than 405 nm.

9. The holographic recording composition according to claim 1, further comprising a photopolymerization initiator.

10. The holographic recording composition according to claim 9, wherein the photopolymerization initiator is a compound denoted by general formula (II). 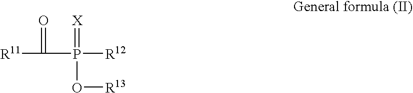
In general formula (II), each of R¹¹, R¹², and R¹³ independently denotes an alkyl group, aryl group, or heterocyclic group, and X denotes an oxygen atom or sulfur atom.

11. A holographic recording medium comprising a recording layer, wherein the recording layer comprises a compound denoted by general formula (I). 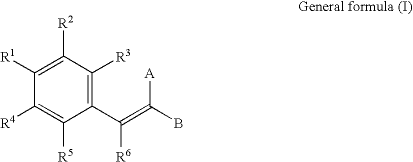
In general formula (I), R¹ denotes a hydrogen atom or a substituent having a Hammett value, σp, of equal to or greater than −0.30, each of R², R³, R⁴, R⁵, and R⁶ independently denotes a hydrogen atom or a substituent, each of A and B independently denotes a substituent and a combination of A and B is one of Combinations 1 to 9, and at least one of R¹, R², R³, R⁴, R⁵, R⁶, A, and B comprises a polymerizable group.

| | | |
|---|---|---|
| (Combination 1) | A: cyano group, | B: cyano group; |
| (Combination 2) | A: oxycarbonyl group, | B: oxycarbonyl group; |
| (Combination 3) | A: acyl group, | B: acyl group; |
| (Combination 4) | A: sulfonyl group, | B: sulfonyl group; |
| (Combination 5) | A: cyano group, | B: acyl group; |
| (Combination 6) | A: cyano group, | B: sulfonyl group; |
| (Combination 7) | A: oxycarbonyl group, | B: acyl group; |
| (Combination 8) | A: oxycarbonyl group, | B: sulfonyl group; |
| (Combination 9) | A: sulfonyl group, | B: acyl group. |

12. The holographic recording medium according to claim 11, wherein, in general formula (I), R¹ denotes a hydrogen atom, alkyl group, aryl group, heterocyclic group, acyl group, sulfonyl group, acyloxy group, alkoxycarbonyl group, aryloxycarbonyl group, alkoxy group, aryloxy group, acylamino group, sulfonylamino group, cyano group, or halogen group, each of R², R³, R⁴, and R⁵ independently denotes a hydrogen atom, alkyl group, aryl group, heterocyclic group, acyl group, sulfonyl group, acyloxy group, alkoxycarbonyl group, aryloxycarbonyl group, alkoxy group, aryloxy group, amino group, acylamino group, sulfonylamino group, or halogen group, and R⁶ denotes a hydrogen atom or an alkyl group.

13. The holographic recording medium according to claim 11, wherein, in general formula (I), R¹ denotes an alkyl group, aryl group, alkoxycarbonyl group, acyloxy group, or halogen group.

14. The holographic recording medium according to claim 11, wherein, in general formula (I), each of R², R³, R⁴, and R⁵ independently denotes a hydrogen atom, alkyl group, aryl group, alkoxy group, aryloxy group, amino group, acyl group, acyloxy group, or halogen group.

15. The holographic recording medium according to claim 11, wherein, in general formula (I), the combination of A and B is Combination 1, 2, 3, 4, 5, 7, or 8.

16. The holographic recording medium according to claim 11, wherein the polymerizable group is a radical polymerizable group.

17. The holographic recording medium according to claim 11, wherein the compound denoted by general formula (I) has a molar absorbance coefficient of equal to or smaller than 200 mol·l·cm⁻¹ at a wavelength of 405 nm.

18. The holographic recording medium according to claim 11, wherein the compound denoted by general formula (I) has a maximum absorption wavelength of shorter than 405 nm.

19. The holographic recording medium according to claim 11, wherein the recording layer comprises a photopolymerization initiator.

20. The holographic recording medium according to claim 19, wherein the photopolymerization initiator is a compound denoted by general formula (II). 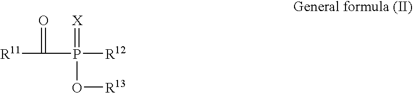
In general formula (II), each of R¹¹, R¹², and R¹³ independently denotes an alkyl group, aryl group, or heterocyclic group, and X denotes an oxygen atom or sulfur atom.
